(19) United States
(12) Patent Application Publication
(10) Pub. No.: US 2024/0067931 A1
(43) Pub. Date: Feb. 29, 2024

(54) METHODS AND COMPOSITIONS FOR SMALL MOLECULE BASED PANCREATIC BETA CELL INDUCTION

(71) Applicant: Trailhead Biosystems Inc., Cleveland, OH (US)

(72) Inventor: Michael BUKYS, Lakewood, OH (US)

(21) Appl. No.: 18/237,265

(22) Filed: Aug. 23, 2023

Related U.S. Application Data
(60) Provisional application No. 63/400,349, filed on Aug. 23, 2022.

(51) Int. Cl. C12N 5/071

(52) U.S. Cl. CPC C12N 5/0676; C12N 2501/999; C12N 2506/45; C12N 2533/50

(57) ABSTRACT

Methods and compositions for generating human pancreatic beta cells, and the progenitor cells thereof, from human pluripotent stem cells using chemically defined culture media are disclosed. The methods allow for the initial generation of FOXA2+HNF1b+ dorsal foregut endodermal cells (DFECs), which can be further differentiated to human PTF1A+PDX1+ pancreatic progenitor cells (PPCs) and human INS+PDX+ pancreatic beta cells (PBCs) according to the methods provided. Culture media, isolated cell populations and kits are also provided.

### RELATED APPLICATION

This application claims priority to U.S. Provisional Application No. 63/400,349, filed Aug. 23, 2022, the entire contents of which is hereby incorporated by reference.

### BACKGROUND OF THE INVENTION

Successful application of Tissue Engineered Medical Products (TEMPs) crucially depends on the generation of highly specialized cell types. Diseases that can potentially be treated or cured using TEMPs include diabetes, heart disease and neuronal degenerative diseases. A common strategy relies on instructing stem cells to differentiate towards the desired cell type through the process of directed differentiation. Conceptually, such attempts seek to provide cellular signals designed to imitate naturally occurring developmental cues and the differentiation process occur through discrete stages. Currently, such methods are developed through tedious testing, where the combinatorial space of plausible signaling inputs are inadequately explored, and the processes rarely consider cost of consumables/reagents. For instance, protein-type agonists are commonly used to imitate natural developmental cues but cost 100-1000-fold more than small molecule agonists or antagonists. Proteins also degrade over time, decreasing their overall efficacy and requiring greater QC requirements for a production process. The high cost is further amplified with each intermediate stage generated and the overall duration of the differentiation protocol. Many of the directed differentiation protocols that have been developed also suffer from poor robustness with only a percentage of the culture attaining the desired phenotype.

Current pancreatic beta cell (PBC) protocols use 6 discrete stages and can take as long as 34 days to generate beta cells that have highly variable functionality (Pagliuca et al., 2014; Rezania et al., 2014; and Velazco et al., 2020). In spite of the foregoing, PBC development has enormous potential in future health care. Specifically, TEMPs derived from PBC protocols (i.e., stem-cell derived insulin producing cells) are currently entering clinical trials. For example, Vertex, which acquired SEMMA, the origin of TEMPs, received accelerated Phase 1/2 approval (NCT04786262) for commencement of a stem cell-based therapy for metabolically unstable Type I diabetes (enrolling 17 patients).

Accordingly, while some progress has been achieved, there remains a need for efficient and robust methods and compositions for generating human pancreatic beta cells and their progenitor cells from human pluripotent stem cells.

### SUMMARY OF THE INVENTION

This disclosure provides methods of generating human pancreatic beta cells (PBCs), including human dorsal foregut endodermal cells (DFECs) and human pancreatic progenitor cells (PPCs), in a three-stage protocol that can be completed in as little as 16 days. The methods use chemically defined culture media that allows for generation of DEFCs within three days of culture, PPCs within six days of culture, and PBCs within 16 days of culture. The defined culture media used to obtain the different types of progenitor cells comprises small molecule agents that either agonize or antagonize particular signaling pathways in the pluripotent stem cells such that differentiation along the endoderm lineage is promoted, leading to cellular maturation and expression of PBC-associated biomarkers. The methods of the disclosure use culture media for differentiation utilizing different components than used in earlier protocols and avoiding the need for certain components required in other protocols. The methods of the disclosure also have the advantage that use of small molecule agents in the culture media allows for precise control of the culture components, involving less differentiation stages, and less time for differentiation to PBCs compared to prior art protocols.

Accordingly, in one aspect, the disclosure pertains to a method of generating human dorsal foregut endodermal cells (DFECs) comprising: culturing human pluripotent stem cells (PSCs) in a culture media comprising a BMP pathway antagonist, an RA pathway agonist, a TGF-β pathway antagonist, and an MEK pathway antagonist on days 0-3 to obtain human DFECs.

The method can comprise further culturing the human DFECs on days 4-6 in a culture media comprising a BMP pathway antagonist, a RA pathway agonist, a TGF-β pathway antagonist, a TAK1 pathway antagonist, a bFGF mimetic, and an Akt pathway antagonist to obtain human pancreatic progenitor cells (PPCs).

The method can then further comprise culturing the human PPCs on days 7-16 in a culture media comprising a Notch pathway inhibitor (e.g., γ-secretase inhibitor), a TGF-β pathway antagonist, and a flavonoid to obtain human pancreatic beta cells (PBCs).

In one embodiment, the human pluripotent stem cells are induced pluripotent stem cells (iPSCs). In another embodiment, the human pluripotent stem cells are embryonic stem cells. In another embodiment, the human pluripotent stem cells are attached to vitronectin-coated plates during culturing.

In another embodiment, the BMP pathway antagonist is selected from the group consisting of LDN193189, DMH1, DMH2, Dorsomorphin, K02288, LDN214117, LDN212854, folistatin, ML347, Noggin, and combinations thereof. In one embodiment, the BMP pathway antagonist is present in the culture media at a concentration within a range of 100-400 nM. In another embodiment, the BMP pathway antagonist is LDN193189, which is present in the culture media at a concentration of 250 nM.

In another embodiment, the RA pathway antagonist is selected from the group consisting of retinoic acid (RA), TTNPB, AM 580, CD 1530, CD 2314, Ch 55, BMS 753, Tazarotene, Isotretinoin, AC 261066, Sr11237, adapalene, EC23, 9-cis retinoic acid, 13-cis retinoic acid, 4-oxo retinoic acid, All-trans Retinoic Acid (ATRA). In one embodiment, the RA pathway antagonist is present in the culture media at a concentration within a range of 0.5-4 nM. In another embodiment, the RA pathway antagonist is RA, which is present in the culture media at a concentration of 2 μM.

In another embodiment, the TGF-β pathway antagonist is selected from the group consisting of A8301, SB-431542, GW788388, SB525334, TP0427736, RepSox, SD-208, and combinations thereof. In one embodiment, the TGFβ pathway antagonist is present in the culture media at a concentration within a range of 200-1000 nM. In another embodiment, the TGFβ pathway antagonist is A8301, which is present in the culture media at a concentration of 500 nM.

In another embodiment, the MEK pathway antagonist is selected from the group consisting of PD0325901, Binimetinib (MEK162), Cobimetinib (XL518), Selumetinib, Trametinib (GSK1120212), CI-1040 (PD-184352), Refametinib, ARRY-142886 (AZD-6244), PD98059, U0126, BI-847325, RO 5126766, and combinations thereof. In one embodiment, the MEK pathway antagonist is present in the culture media at a concentration within a range of 200-1000 nM. In another embodiment, the MEK pathway antagonist is PD0325901, which is present in the culture media at a concentration of 250 nM.

In another embodiment, the TAK1 pathway antagonist is selected from the group consisting of Taki ((5Z)-7-Oxozeaenol), Takinib, Dehydoabietic acid, NG25, Sarsasapogenin, and combinations thereof. In one embodiment, the TAK1 pathway antagonist is present in the culture media at a concentration within a range of 200-1000 nM. In another embodiment, the TAK1 pathway antagonist is Taki ((5Z)-7-Oxozeaenol), which is present in the culture media at a concentration of 500 nM.

In another embodiment, the bFGF mimetic is present in the culture media at a concentration within a range of 150-600 nM. In one embodiment, the bFGF mimetic is SUN11602, which is present in the culture media at a concentration of 300 nM.

In another embodiment, the AKT pathway antagonist is selected from the group consisting of AT7867, MK2206, GSK690693, Perifosine (KRX-0401), Ipatasertib (GDC-0068), Capivasertib (AZD5363), PF-04691502, Triciribine (NSC154020), ARQ751, Miransertib (ab235550), Borussertib, Cerisertib, and combinations thereof. In one embodiment, the AKT pathway antagonist is present in the culture media at a concentration within a range of 100-400 nM. In another embodiment, the AKT pathway antagonist is AT7867, which is present in the culture media at a concentration of 250 nM.

In another embodiment, the Notch pathway antagonist is selected from the group consisting of GSI-XX, R04929097, Semagacestat, Dibenzazepine, LY411575, Crenigacestat, IMR-1, IMR-1A, FLI-06, DAPT, Valproic acid, YO-01027, CB-103, Tangeretin, BMS-906024, Avagacestat, Bruceine D, and combinations thereof. In one embodiment, the Notch pathway antagonist is present in the culture media at a concentration within a range of 50-200 nM. In another embodiment, the Notch pathway antagonist is GSI-XX, which is present in the culture media at a concentration of 100 nM.

In another embodiment, the flavonoid is selected from the group consisting of quercetin, quercetin analogues, (e.g., dihydroquercetin, 6,2′,4′,5′-pentahydroxyflavone, quercetin-3-O-propionate (Q-pr), quercetin-3-O-butyrate (Q-bu), quercetin-3-O-valerate, or 3,4′-Di-O-methylquercetin), genistein, anthocyanins, catechins, gallocatechins (e.g., epigallocatechin-3-gallate (EGCG)), anthocyanidins, apigenin, luteolin, kaemferol, curcumin, myricetin, daidzein, naringin, rutin, and hesperitin. present in the culture media at a concentration within a range of 3-50 μM. In another embodiment, the flavonoid is quercetin, which is present in the culture media at a concentration of 15 μM.

In another aspect, the disclosure provides a method of generating human DFE cells comprising culturing human pluripotent stem cells in a culture media comprising a BMP pathway antagonist, a RA pathway agonist, a TGF-β pathway antagonist, and an MEK pathway antagonist on days 0-3 to obtain human DFE cells.

In another embodiment, the BMP pathway antagonist is LDN193189, the RA pathway agonist is retinoic acid, the TGF-β pathway antagonist is A8301, and the MEK pathway antagonist is PD0325901.

In another embodiment, LDN193189 is present in the culture media at a concentration within a range of 100-400 nM, retinoic acid is present in the culture media at a concentration within a range of 0.4-4 μM, A8301 is present in the culture media at a concentration within a range of 200-1000 nM, and PD0325901 is present in the culture media at a concentration within a range of 100-400 nM.

In another embodiment, LDN193189 is present in the culture media at a concentration of 250 nM, retinoic acid is present in the culture media at a concentration of 2 μM, A8301 is present in the culture media at a concentration of 500 nM, and PD0325901 is present in the culture media at a concentration of 250 nM.

In another aspect, the disclosure provides a method of generating human pancreatic progenitor cells (PPCs) comprising:
(a) culturing human pluripotent stem cells in a culture media comprising a BMP pathway antagonist, a RA pathway agonist, a TGF-β pathway antagonist, and an MEK pathway antagonist on days 0-3 to obtain human dorsal foregut endoderm cells (DFECs); and
(b) further culturing the DFECs on days 4-6 in a culture media comprising a BMP pathway antagonist, a RA pathway agonist, a TGF-β pathway antagonist, a TAK1 pathway antagonist, a bFGF mimetic, and an Akt pathway antagonist to obtain human pancreatic progenitor cells (PPCs).

In one embodiment, the BMP pathway antagonist is LDN193189, the RA pathway agonist is retinoic acid, the TGF-β pathway antagonist is A8301, the MEK pathway antagonist is PD0325901, the TAK1 pathway antagonist is Taki ((5Z)-7-Oxozeaenol), the bFGF mimetic is SUN11602, and the Akt pathway antagonist is AT7867.

In another embodiment, LDN193189 is present in the culture media at a concentration within a range of 100-400 nM, retinoic acid is present in the culture media at a concentration within a range of 0.5-4 μM, A8301 is present in the culture media at a concentration within a range of 200-1000 nM, PD0325901 is present in the culture media in step (a) at a concentration within a range of 100-400 nM, Taki ((5Z)-7-Oxozeaenol) is present in the culture media at a concentration within a range of 200-1000 nM, SUN11602 is present in the culture media at a concentration within a range of 150-600 nM, and AT7867 is present in the culture media in step (a) at a concentration within a range of 100-400 nM.

In another embodiment, LDN193189 is present in the culture media at a concentration of 250 nM, retinoic acid is present in the culture media at a concentration of 2 μM, A8301 is present in the culture media at a concentration of 500 nM, PD0325901 is present in the culture media at a concentration of 250 nM, Taki ((5Z)-7-Oxozeaenol) is present in the culture media at a concentration of 500 nM, SUN11602 is present in the culture media at a concentration of 300 nM, and AT7867 is present in the culture media in step (a) at a concentration of 250 nM.

In yet another aspect, the disclosure provides a method of generating human pancreatic beta cells (PBCs) comprising:
(a) culturing human pluripotent stem cells (hPSCs) in a culture media comprising a BMP pathway antagonist, a RA pathway agonist, a TGF-β pathway antagonist, and an MEK pathway antagonist on days 0-3 to obtain human dorsal foregut endoderm cells (DFECs);
(b) further culturing the DFECs on days 4-6 in a culture media comprising a BMP pathway antagonist, a RA pathway agonist, a TGF-β pathway antagonist, a TAK1 pathway antagonist, a bFGF mimetic, and an Akt pathway antagonist to obtain pancreatic progenitor cells (PPCs); and
(c) further culturing the human PPCs on days 7-16 in a culture media comprising a Notch pathway antagonist, a TGF-β pathway antagonist, and a flavonoid to obtain human pancreatic beta cells (PBCs).

In one embodiment, the BMP pathway antagonist is LDN193189, the RA pathway agonist is retinoic acid, the TGF-β pathway antagonist is A8301, the MEK pathway antagonist is PD0325901, the TAK1 pathway antagonist is Taki ((5Z)-7-Oxozeaenol), the bFGF mimetic is SUN11602, the Akt pathway antagonist is AT7867, the Notch pathway antagonist is 7-secretase inhibitor XX (GSI-XX).

In another embodiment, LDN193189 is present in the culture media in steps (a) and (b) at a concentration within a range of 100-400 nM, retinoic acid is present in the culture media in steps (a) and (b) at a concentration within a range of 0.5-2 μM, A8301 is present in the culture media in steps (a)-(c) at a concentration within a range of 200-1000 nM, PD0325901 is present in the culture media in step (a) at a concentration within a range of 100-400 nM, Taki ((5Z)-7-Oxozeaenol) is present in the culture media in step (b) at a concentration within a range of 200-1000 nM, SUN11602 is present in the culture media in step (b) at a concentration within a range of 150-600 nM, AT7867 is present in the culture media in step (b) at a concentration within a range of 100-400 nM, γ-secretase inhibitor XX (GSI-XX) is present in the culture media in step (c) at a concentration within a range of 50-200 nM, and quercetin is present in the culture media in step (c) at a concentration within a range of 5-30 μM.

In another embodiment, LDN193189 is present in the culture media in steps (a) and (b) at a concentration of 250 nM, retinoic acid is present in the culture media in steps (a) and (b) at a concentration of 2 μM, A8301 is present in the culture media in steps (a)-(c) at a concentration of 500 nM, PD0325901 is present in the culture media in step (a) at a concentration of 250 nM, Taki ((5Z)-7-Oxozeaenol) is present in the culture media in step (b) at a concentration of 500 nM, SUN11602 is present in the culture media in step (b) at a concentration of 300 nM, AT7867 is present in the culture media in step (b) at a concentration of 250 nM, GSI-XX is present in the culture media in step (c) at a concentration of 100 nM, and quercetin is present in the culture media in step (c) at a concentration of 15 μM.

The methods and compositions of the disclosure are useful in the generation of pancreatic beta cells or progenitor cells thereof for use in clinical therapy, research, development, and commercial purposes. For therapeutic applications, the in vitro generated PBCs of the present invention can be administered directly or systemically to a subject for treating or preventing type 1 diabetes, type 2 diabetes, pre-diabetes, conditions due to significant trauma (i.e., damage to the pancreas or loss or damage to islet beta cells), or other metabolic diseases or disorders associated with a deficiency in beta cell number (e.g., a reduction in the number of pancreatic cells), an insufficient level of beta cell biological activity (e.g., a deficiency in glucose-stimulated insulin secretion, or a deficiency in insulin production).

Other features and advantages of the invention will be apparent from the following detailed description and claims.

### BRIEF DESCRIPTION OF THE DRAWINGS

FIG. 1 is a schematic diagram of a representative culture method of the disclosure utilizing a three-stage protocol for generating pancreatic beta cells (PBCs) from human induced-pluripotent stem cells (hiPSCs). The method allows for the initial generation of FOXA2+HNF1b+ dorsal foregut endodermal cells (DFECs), which upon further culturing allows for the generation of human PTF1A+PDX1+ pancreatic progenitor cells (PPCs) and subsequently the generation of human INS+PDX+ pancreatic beta cells (PBCs).

FIG. 2 Optimization of HNF1B from the iPSC-line CR01. A culture of the iPSC cell line Cr01 was exposed to an HD-DoE perturbation matrix composed of compounds regulating the BMP, retinoid, TGF-β and FGF pathways. Pathways previously shown to be involved in the differentiation of dorsal foregut endoderm were utilized in this analysis. MODDE software was used to predict the optimal condition for maximizing HNF1b activation. Predicted factor additives for this optimization and the relative expression under these conditions for the genes measured are shown.

FIG. 3 Optimization of FOXA2 from the iPSC-line CR01. A culture of the iPSC cell line Cr01 was exposed to an HD-DoE perturbation matrix composed of compounds regulating the BMP, retinoid, TGF-β and FGF pathways. Pathways previously shown to be involved in the differentiation of dorsal foregut endoderm. MODDE software was used to predict the optimal condition for maximizing FOXA2 activation. Predicted factor additives for this optimization and the relative expression under these conditions for all genes measured are shown.

FIG. 4 Dual optimization of FOXA2 and HNF1B generates dorsal foregut endoderm (DFE). A culture of the iPSC cell line Cr01 was exposed to an HD-DoE perturbation matrix composed of compounds regulating the BMP, retinoid, TGF-β and FGF pathways. Pathways previously shown to be involved in the differentiation of dorsal foregut endoderm were evaluated. MODDE software was used to predict the optimal condition for maximizing FOXA2 activation. Predicted factor additives for this optimization and the relative expression under these conditions for all genes measured are shown.

FIG. 5 Dynamic profiling demonstrates the requirement for MEK and TGF-β inhibition for FOXA2 induction. A culture of the iPSC cell line Cr01 was exposed to an HD-DoE perturbation matrix composed of compounds regulating the BMP, retinoid, TGF-β and FGF pathways. Use of MODDE software to visualize the dynamic profiling shows the relative contributions of the critical effectors.

FIG. 6 Coefficient plots show main effectors needed for DFE differentiation. A culture of the iPSC cell line Cr01 was exposed to an HD-DoE perturbation matrix composed of compounds regulating the BMP, retinoid, TGF-β and FGF pathways. Coefficient plots are shown for the FOXA2 and HNF1B genes. The magnitude and direction of the bars are indicative of the overall contribution of the factors to gene activation or repression, where positive bars indicate contributions to activation and negative bars indicate contributions to inhibition.

FIG. 7 A heat map confirms the critical input components needed for DFE generation. Duplicate samples of iPSC (CR01) cultures were exposed to media containing different combinations in which retinoic acid, LDN193189, A8301, PD0325 and/or Wnt3a were added to the cultures. The combination used is indicated at the top of the heat map. Samples had RNA purifications were performed on the samples and were sent for sequencing. Genes representative of different endoderm regions were monitored and relative levels of these transcripts are shown. A number of genes previously identified as contributing to the suppression of neural fates are shown towards the bottom of the heat map. Tissue regions corresponding the genes are shown on the left side of the heat map.

FIG. 8 DFE is directly induced without going through a primitive streak or a definitive endoderm intermediate. Duplicate samples of iPSC (CR01) cultures were exposed to media containing different combination of the additive retinoic acid, LDN193189, A8301, PD0325 and/or Wnt3a. Combinations used are indicated at the top of the heat map. Samples had RNA purifications were performed on the samples and were sent for sequencing. Genes representative of the primitive streak or of a generalized definitive endodermal state have their relative expression levels shown on the left side of the heatmap.

FIG. 9 Achieving optimal FOXA2 expression. IPSC (CR01) cultures were exposed to media containing different combination of the additive retinoic acid, LDN193189, A8301 and/or PD0325 as indicated on the left side of the stainings. After three days, cultures were fixed and subjected to immunohistochemical staining for the protein antigens of FOXA2 and HNF1b.

FIG. 10 Dynamic profiling demonstrates pancreatic potential. Dynamic profiling of a combination of genes known to mark the location of the future pancreatic field is shown. This was done on the HD-Doe Design shown in Examples 1-3.

FIG. 11 Modelling for maximal PDX1 induction. Cultures of the iPSC cell line Cr01 was differentiated to a DFE state and then subjected to an HD-DoE perturbation matrix composed of the compounds indicated in the figure. MODDE software was used to predict the optimal condition for maximizing PDX1 activation. Predicted factor additives for this optimization and the relative expression under these conditions for the genes measured are shown.

FIG. 12 Coefficient plots show main effectors for pancreatic progenitor generation from a DFE precursor. Coefficients for the HD-DoE design shown in Example 9 are shown for PDX1 and PTF1A. The co-expression of these two genes is indicative of a pancreatic field.

FIG. 13 Optimizing for PDX1 induction. IPSC cultures were differentiated to DFE cultures and then subjected to the two opposing PDX1 optimizers. ESC CPP^{opt} refer to the conditions previously identified (Bukys et al 2020) and are the conditions identified in Example 10. Cultures were differentiated for an additional 3 days in these conditions before being fixed with formalin and stained with antibodies recognizing the protein transcription factors PDX1 and NKX6.1. The co-expression of PDX1 and NKX6.1 is indicative of a pro-endocrine pancreatic progenitor.

FIG. 14 Dynamic profiling exploring endocrine cell sub-types. IPSC cultures were differentiated to DFE cultures and then subjected a HD-DoE matrix as outlined in Example 10. MODDE software was used to display the dynamic profiles of GCG, INS and SST, which provide a representable characterization of the different pancreatic endocrine subtypes corresponding to alpha, beta, and delta cells, respectively.

FIG. 15 Modelling for maximal insulin expression. Cultures of the iPSC cell line CR01 were differentiated to a DFE state and then subjected to an HD-DoE perturbation matrix composed of the compounds indicated in the figure. The cultures were then incubated for an additional 5 days in the presence of γ-secretase inhibitor XX (GSI-XX) and A8301. These are compounds known to induce endocrine commitment from a pancreatic progenitor. MODDE software was used to predict the optimal condition for maximizing PDX1 activation. Predicted factor additives for this optimization and the relative expression levels under these conditions for the genes measured are shown.

FIG. 16 Dynamic profiling of endocrine cell sub-types. Cultures of the iPSC cell line CR01 were differentiated to a DFE state and then subjected to an HD-DoE perturbation matrix composed of the compounds indicated in the figure. These cultures were then incubated for an additional 5 days in the presence of GSI-XX and A8301. These are compounds known to induce endocrine commitment from a pancreatic progenitor. MODDE software was used to display the dynamic profiles of GCG, INS and SST, which provide a representable characterization of the different pancreatic endocrine subtypes corresponding to alpha, beta, and delta cells, respectively.

FIG. 17 Immunohistochemical validation of de novo beta cells. Cultures of the iPSC cell line CR01 were differentiated to a DFE state for 3 days. These cultures were then incubated in the presence of stage 2 media for an additional 3 days and then incubated for an additional 10 days in the presence media containing GSI-XX and A8301. Cultures were then fixed with formalin and stained used antibodies that recognize the proteins PDX1/Insulin or PDX1/C-Peptide as indicated in FIG. 17.

FIG. 18 HD-DoE screening for optimal de novo beta cell induction. Culture of the iPSC cell line CR01 were differentiated to a DFE state for 3 days. These cultures were then incubated in the presence of stage 2 media for an additional 3 days. These cultures were then incubated for an additional 5 days in the presence of GSI-XX and A8301 followed by a 3 day incubation in the presence of a perturbation matrix composed of the compounds indicated in the different panels. This was performed 3 separate times (once for each of the 3 panels). These HD-DoE experiments were then analyzed using MODDE software for maximal induction of insulin. The predicted results are shown.

FIG. 19 Heat map showing descending factor contributions for the induction of INS. The three HD-DoE experiments shown in Example 17 were used for modeling the maximal expression of several other genes, including GCG, SST, PDX1, PAX4, MAFA and NKX2.2, which are indicative of endocrine cell types with a focus on pancreatic beta cells as indicated at the top of the heat map. The relative factor contributions for the activation of these different genes were then used to generate a secondary heat map representative of the gene induction logic and then ranked according to the compound's ability to induce INS. Dark blue colors indicate a high inductive capacity, while dark red colors indicative of a highly repressive capacity for activation of the genes are shown.

FIG. 20 HD-DoE screening for optimal de novo beta cell induction. Cultures of the iPSC cell line CR01 were differentiated to a DFE state for 3 days. These cultures were then incubated in the presence of stage 2 media for an additional 3 days. These cultures were then incubated for an additional 5 days in the presence of GSI-XX and A8301 followed by a 3 day incubation in the presence of a perturbation matrix composed of the compounds indicated in the different panels. This was performed 4 separate times (once for each of the 4 panels). These HD-DoE experiments were then analyzed using MODDE software for the maximal induction of Insulin. The predicted results are shown.

FIG. 21 Heat map showing descending factor contributions for the induction of INS. The four HD-DoE experiments shown in Example 19 were used for modeling the maximal expression of several other genes. These genes were GCG, SST, PDX1, PAX4, MAFA and NKX2.2. These genes are all indicative of endocrine cell types with a focus on the beta cell as indicated at the top of the heatmap. The relative factor contribution for the activation of these different genes were then used to generate a secondary heat map representative of these genes induction logic and then ranked order according to the compounds ability to induce INS. Dark blue indicates high inductive capacity, while dark red is indicative of a highly repressive effect for the activation of the genes shown.

FIGS. 22-25. Dynamic profiling of endocrine sub-type regulation. One of the HD-DoE experiments shown in Example 19 was analyzed using MODDE software and dynamic profiling of the endocrine products GCG, SST and INS are shown.

FIG. 26 Immunohistochemical validation of the generation of the de novo beta cells. Cultures of the iPSC cell line CR01 was differentiated to a DFE state for 3 days. These cultures were then incubated in the presence of stage 2 media for an additional 3 days. These cultures were then incubated for an additional 10 days in the presence of quercetin, GSI-XX and A8301. The cultures were then fixed using formalin and stained for antibodies that recognize the proteins PDX1 and CPEP.

FIG. 27 Immunohistochemical validation of the generation of the de novo beta cells. Culture of the iPSC cell line CR01 were differentiated to a DFE state for 3 days. These cultures were then incubated in the presence of stage 2 media for an additional 3 days, followed by incubation in the presence of quercetin, GSI-XX and A8301 for an additional 10 days. The cultures were then fixed using formalin and stained for antibodies that recognize the proteins CGC/CPEP, PDX1/CPEP or PDX1/SOX9 as indicated.

FIG. 28 Functional analysis of beta cells. Cultures of the iPSC cell line CR01 were differentiated to a DFE state for 3 days. These cultures were then incubated in the presence of stage 2 media for an additional 3 days, followed by incubation in the presence of quercetin, GSI-XX and A8301 for an additional 10 days. This culture washed with PBS followed by a 15-minute incubation in media containing 3 mM glucose (basal media). Each of the medias was then changed to media either containing 17.5 mM glucose or 30 mM KCl as indicated on the X-axis. Samples were run in triplicate. Quantification was performed using a C-peptide ELISA. Samples were normalized to the C-peptide levels detected in the basal media samples.

FIG. 29 Directed differentiation beta cell protocol is amenable to suspension culture systems. The differentiation protocol was performed in PBS vertical wheel bioreactors as indicated in the schematic. Pictures of aggregates from the different stages are shown.

FIG. 30 Growth and expansion occurs throughout the differentiation process. Samples were taken daily from the bioreactors shown in FIG. 29. The samples were quantified on a Cell Countess and graphed using Graphpad prism software.

FIG. 31 Differentiation within a suspension-systems generates similar induction of beta cells. Samples were taken from the bioreactors shown in FIG. 29 at the end of each stage and subjected to RNA isolation. Technical replicates were then subjected to cDNA conversion and relative levels were quantified on a QuantStudio using custom designed chips. The relative expression levels for key genes are shown as compared to levels obtained from a control stage 3. The control stage 3 sample consisted of cells that were differentiated using the same protocol on adherent cultures.

FIG. 32 De novo insulin production within bioreactors. Quantification of samples from stage 3 bioreactors are shown in FIG. 29. Potassium mediated release was performed in cell culture inserts with inserts being moved from basal media to the 30 mM KCl solution. A sample was removed from the bioreactor and the number of aggregates per ml was calculated. This was then centrifuged and a sample of the supernatant was used to quantify the conditioned media. The pellets were dissolved in TPER and then used to quantify the C-peptide content per aggregate. All quantification was performed using C-peptide ELISAs.

FIGS. 33A-B. Bioreactor runs consistently achieve insulin producing cells. FIG. 33A shows schematic diagrams referencing the different stages evaluated through immunohistochemistry (IHC) analysis in panel B. FIG. 33B shows Brightfield images of iPSC aggregates.

FIG. 34. Bioreactor produced endocrine cells have similar expression patterns as compared to human Islets. Aggregates produced within a bioreactor run and primary human islets were seeded onto Matrigel for an IHC analysis comparison. Cells were evaluated for gene expression patterns, which demonstrated similar expression patterns.

FIG. 35. Determining the average insulin content per cell. The bar graph shows results for pg insulin/cell.

FIG. 36. Insulin production continues for up to two weeks after initial induction. The graph shows results for bioreactor cultures monitored and sampled along the course of a stage 3 induction.

FIG. 37. Glucose stimulated insulin secretion is not maintained after cryopreservation. The results in the graph show that the iPSC derivatives demonstrate a variable degree of functionality when challenged with glucose influxes.

FIG. 38. Bioreactor-based production runs consistently produce insulin producing cells at levels comparable to human Islets. RNA sequencing results are from three separate bioreactor runs of stage 3 cells performed to evaluate expression patterns representative of different aspects of the differentiation process.

FIG. 39. Functionality of bioreactor produced insulin secreting cells occurs through cAMP agonism. The bar graph shows results for assaying the mechanism of insulin secretion for the iPSC derivatives, which showed that when cAMP agonist were used in conjunction with a GSIS assay they were able to increase insulin secretion.

FIG. 40. Mature glucose stimulated insulin secretion mechanisms are under-developed in the immature iPSC-derivatives. RNA sequencing results are shown for expression of the indicated genes, demonstrating that the incretin receptor GLPR1 had very low expression in the iPSC-derivatives, glucokinase (GCK) expression was similar between the iPSC-derivatives and primary beta cells and the GLUT2 transporter had lower expression in the iPSC-derivatives.

### DETAILED DESCRIPTION OF THE INVENTION

Described herein are methodologies and compositions that allow for the generation of pancreatic beta cells (PBCs) and their progenitor cells from human pluripotent stem cells under chemically defined culture conditions using a small molecule-based approach. The methods of the disclosure generate PBCs and their progenitors in a three-stage protocol in which FOXA2+HNF1b+ dorsal foregut endodermal cells (DFEs) are generated in three days, followed by generation of PDX1+PTFIA+NKX6.1+ pancreatic progenitor cells (PPCs) by day six of culture, followed by generation of pancreatic beta cells (PBCs) by day 11 of culture. Thus, the disclosure allows for obtention of PBCs in a significantly shorter time than prior art protocols using chemically defined culture conditions.

Various aspects of the invention are described in further detail in the following subsections.

### I. Cells

The starting cells in the cultures are human pluripotent stem cells. As used herein, the term “human pluripotent stem cell” (“hPSC”) refers to a human stem cell that has the capacity to differentiate into a variety of different cell types. The term “pluripotent” as used herein refers to a cell with the capacity, under different conditions, to differentiate to cell types characteristic of all three germ cell layers (endoderm, mesoderm and ectoderm). Pluripotent cells are characterized primarily by their ability to differentiate to all three germ layers, for example, using a nude mouse and teratomas formation assay. Pluripotency can be evidenced by the expression of embryonic stem (ES) cell markers, although the preferred test for pluripotency is the demonstration of the capacity to differentiate into cells of each of the three germ layers.

hPSCs include, for example, induced pluripotent stem cells (iPSC) and human embryonic stem cells, such as ES cell lines. Non-limiting examples of induced pluripotent stem cells (iPSC) include 19-11-1, 19-9-7 or 6-9-9 cells (e.g., as described in Yu, J. et al. (2009) Science 324:797-801). Non-limiting examples of human embryonic stem cell lines include ES03 cells (WiCell Research Institute) and H9 cells (Thomson, J. A. et al. (1998) Science 282:1145-1147). Human pluripotent stem cells (PSCs) express cellular markers that can be used to identify cells as being PSCs. Non-limiting examples of pluripotent stem cell markers include TRA-1-60, TRA-1-81, TRA-2-54, SSEA1, SSEA3, SSEA4, CD9, CD24, OCT3, OCT4, NANOG and/or SOX2. Since the methods for generating the progenitor populations of the disclosure are under differentiation from the starting pluripotent stem cell population, in various embodiments the progenitor cell populations generated by the methods of the disclosure lack expression of one or more stem cell markers, including those described in the Examples.

Common practices and methods for coating TC plates or passaging of iPSCs may be used. In exemplary embodiments, PSC cultures, such as CR01 iPSC line, can be maintained and grown on vitronectin coated 6-well tissue culture (TC) plates. E8 media may be used for general maintenance of these cells. PSC cultures are generally passaged every 3-4 days using EDTA to disrupt cell-to-cell adhesion. This can be accomplished by removing the E8 media and washing each well of the TC plate with 2 ml of PBS. A 3-minute incubation in the presence of 5 mM EDTA can then be performed at 37 degrees C. Wells are then aspirated and the cells are washed off of the plates and seeded in fresh E8 media supplemented with 1X RevitaCell. Each well passaged is generally seeded onto 6 wells of a newly vitronectin-coated TC plate resulting in a 1 to 6 expansion of the iPSC line.

Passaging of PSCs may be performed using collagenase, accutase, trypsin, TyrPLE or other digestion enzymes instead of EDTA. PSCs can also be maintained and grown on substrates other than vitronectin. Commonly used substrates include gelatin, Matrigel, geltrex or other ECM or charged surface coatings. In addition, other ROCK inhibitors may be used instead of those in the RevitaCell supplement. A commonly used inhibitor is Y27632.

The pluripotent stem cells are subjected to culture conditions, as described herein, that induce cellular differentiation. As used herein, the term “differentiation” refers to the development of a cell from a more primitive stage toward a more mature (i.e., less primitive) cell, typically exhibiting phenotypic features of commitment to a particular cellular lineage.

In some embodiments, the cells generated by the methods of the disclosure are dorsal foregut endodermal cells (DFECs). As used herein, a “dorsal foregut endodermal cell” or “DFEC” refers to a cell that is more differentiated than a pluripotent stem cell in that it is committed to the endoderm lineage but still has the capacity to differentiate into different types of cells along that same lineage. The DFEC expresses the biomarkers FOXA2 and HNF1b.

The DFEC may also express additional biomarkers, including but not limited to the endodermal region markers of PROM1, CPB1, ONECUT1, and CXCL4; the endodermal dorsal markers of SFRP5, MNX1, PTCH1 and PAX6; and the midgut markers FOXA2, HNF1b, HOXA3 and ONECUT2. In contrast, the DFECs exhibit very low or undetectable expression of the ventral markers HHEX and NR5A2 and the classically defined definitive endodermal markers SOX17, GSC, MIXL1 and CER.

In some embodiments, the cells generated by the methods of the disclosure are pancreatic progenitor cells (PPCs), which are more differentiated (more mature) cells than DFECs and are committed to particular cell types within the endoderm lineage. A PPC of the present invention expresses the biomarkers PTF1A and PDX1.

The committed PPCs generated by the methods of the disclosure can be further cultured in vitro to generate mature human pancreatic beta cells (PBCs). As used herein, a “pancreatic beta cell” or “PBC” refers to a stem cell-derived pancreatic beta cell that expresses the biomarkers INS and PDX.

In some embodiments, cells can be identified and characterized based on expression of one or more biomarkers specific to or characteristic of DFECs, PPCs, or PBCs. A “positive” biomarker is one that is expressed on a cell of interest, whereas a “negative” biomarker is one that is not expressed on a cell of interest. In the embodiments described herein, the DFEC is FOXA2+HNF1b+, the PPC is PTF1A+PDX1+ and the PBC is INS+PDX+.

As used herein, expression by a cell of only “low” levels of a biomarker of interest is intended to refer to a level that is at most 20%, and more preferably, less than 20%, less than 15%, less than 10% or less than 5% above background levels (wherein background levels correspond to, for example, the level of expression of a negative control marker that is considered to not be expressed by the cell).

### II. Culture Media Components

The methods of the disclosure for generating DFECs, PPCs, and PBCs comprise culturing human pluripotent stem cells in culture media comprising specific agonists and/or antagonists of cellular signaling pathways. In some embodiments, the culture media lacks serum, lacks exogenously added growth factors, lacks animal products, is serum-free, is xeno-free and/or is feeder layer free.

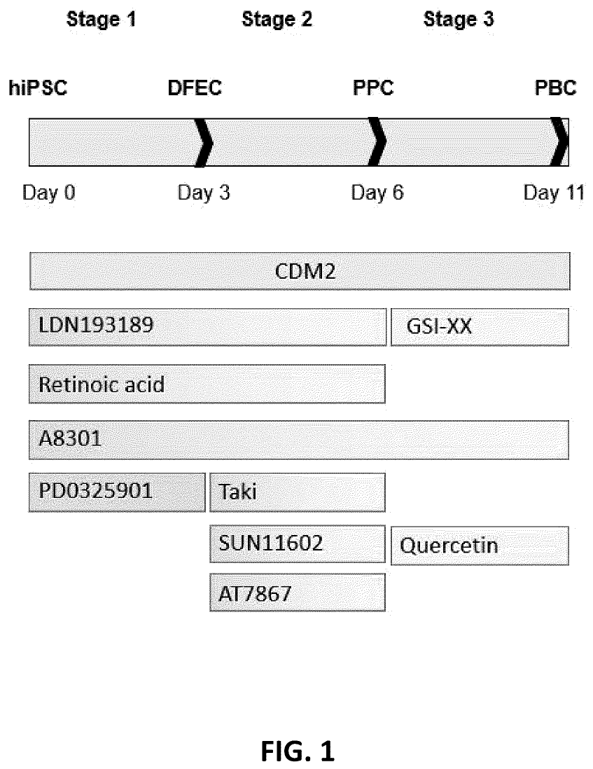

As shown in the exemplary embodiment depicted in FIG. 1, a culture media comprising a BMP pathway antagonist (e.g., LDN193189), an RA pathway agonist (e.g., RA), a TGF-β pathway antagonist (e.g., A8301), and a MEK pathway antagonist (e.g., PD032901) was sufficient to generate foregut endodermal cells (DFECs) in as little as three days (referred to herein as “stage 1” of the differentiation protocol). In some embodiments, the pluripotent cells may be cultured for up to 5 days in stage 1 of the differentiation protocol.

Further differentiation of the DFECs to pancreatic progenitor cells (PPC) can be achieved after three more days (referred to herein as “stage 2”) by culturing the DEFCs in a culture media comprising a BMP pathway antagonist, such as LDN193189; an RA pathway agonist, such as retinoic acid (RA); a TGF-β pathway antagonist, such as A8301; a TAK1 pathway antagonist, such as Taki; a bFGF mimetic, such as SUN11602; and an Akt pathway antagonist, such as AT7867. In some embodiments, the DFECs may be cultured for up to 5 days in stage 2 of the differentiation protocol.

Still further differentiation of the PPCs to pancreatic beta cells (PBCs) can be achieved in in as little as 5 days (referred to as “stage 3”) by culturing the PPCs in a culture media comprising a Notch pathway antagonist, such as γ-secretase inhibitor XX (GSI-XX); a TGF-β pathway antagonist, such as A8301; and a flavonoid, such as quercetin or a quercetin analogue thereof. In some embodiments, the DFECs can be cultured for up to 20 days in stage 3 culture media of the differentiation protocol.

As used herein, an “agonist” of a cellular signaling pathway is used with reference to an agent that stimulates (upregulates) the cellular signaling pathway. In some embodiments, stimulation of the cellular signaling pathway can be initiated extracellularly, for example by use of an agonist that activates a cell surface receptor involved in the signaling pathway (e.g., the agonist can be a receptor ligand). Additionally, or alternatively, stimulation of cellular signaling can be initiated intracellularly, for example, by use of a small molecule agonist that interacts intracellularly with one or more component(s) of the signaling pathway.

As used herein, an “antagonist” of a cellular signaling pathway is used with reference to an agent that inhibits (downregulates) the cellular signaling pathway. In some embodiments, inhibition of the cellular signaling pathway can be initiated extracellularly, for example by use of an antagonist that blocks a cell surface receptor involved in the signaling pathway. Additionally, or alternatively, inhibition of cellular signaling can be initiated intracellularly, for example, by use of a small molecule antagonist that interacts intracellularly with one or more component(s) of the signaling pathway.

Agonists and antagonists used in the methods of the disclosure are known and/or are commercially available. They are used in the culture media at concentrations effective to achieve the desired outcome, e.g., generation of DFEs, PPCs, or PBCs, each characterized by specific corresponding markers. Non-limiting examples of suitable agonist and antagonist agents and effective concentration ranges are described further below.

Antagonists of the BMP (bone morphogenetic protein) pathway include agents, molecules, compounds, or substances capable of inhibiting (downregulating) the BMP signaling pathway, which is biologically activated by binding of BMP to a BMP receptor, which is an activin receptor-like kinase (ALK) (e.g., type I BMP receptor, including but not limited to ALK2 and ALK3). In one embodiment, the BMP pathway antagonist is selected from the group consisting of LDN193189, DMH1, DMH2, Dorsomorphin, K02288, LDN214117, LDN212854, follistatin, ML347, Noggin, and combinations thereof. In one embodiment, the BMP pathway antagonist is present in the culture media at a concentration within a range of 100-500 nM, 100-400 nM, 150-350 nM or 200-300 nM. In one embodiment, the BMP pathway antagonist is LDN193189. In another embodiment, the BMP pathway antagonist is LDN193189, which is present in the culture media at a concentration within a range of 100-500 nM, 100-400 nM, 150-350 nM or 200-300 nM. In another embodiment, the BMP pathway antagonist is LDN193189, which is present in the culture media in steps (a) and (b) of the method (i.e., stages 1 and 2) at a concentration of 250 nM.

Agonists of the retinoic acid (RA) pathway include agents, molecules, compounds, or substances capable of activating (upregulating) the RA signaling pathway. In one embodiment, the RA pathway agonist is selected from the group consisting of retinoic acid (RA), TTNPB, AM 580, CD 1530, CD 2314, CD 437, Ch 55, BMS 753, BMS 961, Tazarotene, Tamibarotene, Isotretinoin, Tretinoin, AC 261066, AC 55649, Sr11237, adapalene, EC23, 9-cis retinoic acid, 13-cis retinoic acid, 4-oxo retinoic acid, and All-trans Retinoic Acid (ATRA), AY 9944 dihydrochloride, Ciliobrevin A, Cyclopamine, or combinations thereof. In one embodiment, the RA pathway agonist is present in the culture media at a concentration within a range of 0.05-5 μM, 0.5-5 μM, or 1-3 μM. In another embodiment, the RA pathway agonist is RA, which is present in the culture at a concentration in a range of 0.2-5 μM, 0.5-4 μM, 1-3 μM. In another embodiment, the RA pathway agonist is RA, which is present in the culture media in steps (a)-(b) of the method (i.e., stages 1 and 2) at a concentration of 2 μM.

Antagonists of the TGFβ (transforming growth factor beta) pathway include agents, molecules, compounds, or substances capable of inhibiting (downregulating) signaling through a TGFβ receptor family member, a family of serine/threonine kinase receptors. In one embodiment, the TGFβ pathway antagonist is selected from the group consisting of A8301, SB-431542, GW788388, SB525334, TP0427736, RepSox, SD-208, and combinations thereof. In one embodiment, the TGFβ pathway antagonist is present in the culture media at a concentration within a range of 200-1000 nM, 250-750 nM, 300-650 nM, or 400-600 nM. In one embodiment, the TGFβ pathway antagonist is A8301. In another embodiment, the TGFβ pathway antagonist is A8301, which is present in the culture media at a concentration of 200-1000 nM, 250-750 nM, 300-650 nM, or 400-600 nM. In another embodiment, the TGFβ pathway antagonist is A8301, which is present in the culture media in steps (a)-(c) of the method (i.e., stages 1-3) at a concentration of 500 nM.

Antagonists of the MEK pathway include agents, molecules, compounds, or substances capable of inhibiting (downregulating) the signaling pathway of one or more of the components of the MAPK/ERK pathway (also known as the Ras-Raf-MEK-ERK pathway). In one embodiment, the MEK pathway antagonist is selected from the group consisting of PD0325901, Binimetinib (MEK162), Cobimetinib (XL518), Selumetinib, Trametinib (GSK1120212), CI-1040 (PD-184352), Refametinib, ARRY-142886 (AZD-6244), PD98059, U0126, BI-847325, RO 5126766, and combinations thereof. In one embodiment, the MEK pathway antagonist is present in the culture media at a concentration within a range of 25-1000 nM, 50-750 nM, 75-500 nM, 100-400 nM, or 150-300 nM. In one embodiment, the MEK pathway antagonist is PD0325901. In another embodiment, the MEK pathway antagonist is PD0325901, which is present in the culture media at a concentration within a range of 25-300 nM, 50-150 nM, 50-250 nM, or 150-300 nM. In another embodiment, the MEK pathway antagonist is PD0325901, which is present in the culture media in step (a) of the method (i.e., stage 1) at a concentration of 250 nM.

Antagonists of the TAK1 (also known as MAP3K7) pathway include agents, molecules, compounds, or substances capable of inhibiting (downregulating) signaling through TAK1 (MAP3K7). In one embodiment, the TAK1 pathway antagonist is selected from the group consisting of Taki ((5Z)-7-Oxozeaenol), Takinib, Dehydoabietic acid, NG25, Sarsasapogenin, and combinations thereof. In one embodiment, the TAK1 pathway antagonist is present in the culture media at a concentration within a range of 200-1000 nM, 250-750 nM, 300-650 nM, or 400-600 nM. In another embodiment, the TAK1 pathway antagonist is Taki, which is present in the culture media at a concentration of 300-800 nM, 250-750 nM, 300-650 nM, or 400-600 nM. In another embodiment, the TAK1 pathway antagonist is Taki, which is present in the culture media in step (b) of the method (i.e., stage 2) at a concentration of 500 nM.

bFGF mimetics include agents, molecules, compounds, or substances capable of activating (upregulating) signaling through the fibroblast growth factor 2 (FGF2) signaling pathway. In an embodiment, the bFGF mimetic is SUN11602.

Antagonists of the AKT pathway include agents, molecules, compounds, or substances capable of inhibiting (downregulating) the signaling pathway of one or more of the serine/threonine kinase AKT family members, which include AKT1 (also designated PKB or RacPK), AKT2 (also designated PKBP or RacPK-β) and AKT 3 (also designated PKB7 or thyoma viral proto-oncogene 3). In an embodiment, the AKT pathway antagonist is selected from the group consisting of AT7867, MK2206, GSK690693, Perifosine (KRX-0401), Ipatasertib (GDC-0068), Capivasertib (AZD5363), PF-04691502, Triciribine (NSC154020), ARQ751, Miransertib (ab235550), Borussertib, Cerisertib, and combinations thereof. In an embodiment, the AKT pathway antagonist is present in the culture media at a concentration within a range of 25-1000 nM, 50-750 nM, 75-500 nM, 100-400 nM, or 150-300 nM. In one embodiment, the AKT pathway antagonist is AT7867. In another embodiment, the AKT pathway antagonist is AT7867, which is present in the culture media at a concentration within a range of 25-300 nM, 50-150 nM, 50-250 nM, or 150-300 nM. In another embodiment, the AKT pathway antagonist is AT7867, which is present in the culture media in step (b) of the method (i.e., stage 2) at a concentration of 250 nM.

Notch pathway antagonists include agents, molecules, compounds, or substances capable of inhibiting (downregulating) signaling through or activity of the Notch transcription factor, including gamma secretase inhibitors (GSIs). In an embodiment, the Notch pathway antagonist is selected from the group consisting of GSI-XX, GSI-X, R04929097, Semagacestat, Avagacestat, Dibenzazepine, LY411575, LY450149, DAPT, Crenigacestat, MK0752, BMS-708163, BMS-906024, CB-103, AL101, Compound E, Compound X (CX), IMR-1, IMR-1A, FLI-06, Valproic acid, YO-01027, Tangeretin, Bruceine D, and combinations thereof. In an embodiment, the Notch pathway antagonist is present in the culture media at a concentration within a range of 25-200 nM, 50-150 nM, or 75-125 nM. In another embodiment, the Notch pathway antagonist is GSI-XX. In another embodiment, the Notch pathway antagonist is GSI-XX, which is present in the culture media at a concentration of 25-200 nM, 50-150 nM, or 75-125 nM. In another embodiment, the Notch pathway antagonist is GSI-XX, which is present in the culture media in step (c) of the method (i.e., stage 3) at a concentration of 100 nM.

Flavonoids include agents, molecules, compounds, or substances selected from the group consisting of quercetin, quercetin analogues, (e.g., dihydroquercetin, 6,2′,4′,5′-pentahydroxyflavone, quercetin-3-O-propionate (Q-pr), quercetin-3-O-butyrate (Q-bu), quercetin-3-O-valerate, or 3,4′-Di-O-methylquercetin), genistein, anthocyanins, catechins, gallocatechins (e.g., epigallocatechin-3-gallate (EGCG)), anthocyanidins, apigenin, luteolin, kaemferol, curcumin, myricetin, daidzein, naringin, rutin, and hesperitin. In an embodiment, the flavonoid is quercetin. In one embodiment, the flavonoid is present in the culture media at a concentration within a range of 3-50 μM, 5-30 μM or 10-20 μM. In another embodiment, the flavonoid is quercetin, which is present in the culture media within a range of 3-50 μM, 5-30 μM or 10-20 μM. flavonoid is quercetin, which is present in the culture media at a concentration of 15 μM.

When an agonist or antagonist is used in more than one step of the method, the agonist or antagonist may be the same or different for one or more of the steps in which the agent is present in the culture media. Further, when an agonist or antagonist is used in more than one step of the method, the concentration of the same agonist or antagonist may be the same or different for each step in which the agent is present in the culture media.

### III. Culture Conditions

In combination with the chemically defined and optimized culture media described in subsection II above, the methods of generating DFEs, PPs, and PBCs of the disclosure utilize standard culture conditions established in the art for cell culture. For example, cells can be cultured at 37° C. and under 5% 02 and 5% CO₂ conditions. In one embodiment, cells can be cultured in standard culture vessels or plates, such as 96-well plates. In certain embodiments, the starting pluripotent stem cells are adhered to plates, preferably coated with an extracellular matrix material, such as vitronectin. In one embodiment, the stem cells are cultured on a vitronectin coated culture surface (e.g., vitronectin coated 96-well plates).

Pluripotent stem cells can be cultured in commercially available media prior to differentiation. For example, stem cells can be cultured for at least one day in Essential 8 Flex media (Thermo Fisher #A2858501) prior to the start of the differentiation protocol. In a non-limiting exemplary embodiment, stem cells are passaged onto vitronectin (Thermo Fisher #A14700) coated 96-well plates at a cell density of 150,000 cells/cm² and cultured for one day in Essential 8 Flex media prior to differentiation.

To initiate the differentiation protocol, the media in which the stem cells are cultured is changed to a basal differentiation media that has been supplemented with signaling pathway agonists and/or antagonists as described above in subsection II. A basal differentiation media can include, for example, a commercially available base supplemented with additional standard culture media components needed to maintain cell viability and growth, but lacking serum (the basal differentiation media is a serum-free media) or any other exogenously added growth factors, such as bFGF (FGF2), PDGF or HGF. In a non-limiting exemplary embodiment, a basal differentiation media contains 1× IMDM (Thermo Fisher #12440046), 1× F12 (Thermo Fisher #11765047), poly(vinyl alcohol) (Sigma #p8136) at 1 mg/ml, chemically defined lipid concentrate (Thermo Fisher #11905031) at 1%, 1-thioglycerol (Sigma #M6145) at 450 μM, Insulin (Sigma #11376497001) at 0.7 ug/ml and transferrin (Sigma #10652202001) at 15 ug/ml (also referred to herein as “CDM2” media, as used in the exemplary differentiation protocols shown herein and as depicted in FIG. 1. The culture media typically is changed regularly to fresh media. For example, in one embodiment, media is changed every 24 hours.

To generate DFECs, PPCs, and PBCs, the starting stem cells are cultured in the optimized culture media for sufficient time for cellular differentiation and expression of committed DFE, PPC, or PBC markers. As described in the Examples, it has been discovered that culture of pluripotent stem cells in a three-stage method, one optimized for generation of DFECs, a second optimized for generation of PPCs, and a third optimized for the generation of PBCs so as to produce PBCs in as little as 16 days of culture. The culture period for the first stage (leading to DFECs) corresponds to days 1-3, the culture period for the second stage (leading to PPCs) corresponds to days 4-6, and the culture period for the third stage (leading to PBCs) corresponds to days 7-16.

Accordingly, in the first stage of the method for generating DFECs, also referred to herein as “step (a)” or “stage 1”, pluripotent stem cells are cultured in the stage 1-optimized culture media on days 0-3, or starting on day 0 and continuing through day 3, or for 72 hours (3 days), or for at least 60 hours, or at least 64 hours, or at least 68 hours, or at least 70 hours, or for 60 hours, or for 64 hours, or for 68 hours, or for 70 hours, or for 72 hours. In some embodiments, the pluripotent cells may be cultured for up to 5 days in stage 1 of the differentiation protocol. Therefore, in certain embodiments, the pluripotent stem cells may be cultured in the stage 1-optimized culture media for 72 hours followed by: at least 8 additional hours, at least 8 additional hours, at least 16 additional hours, at least 24 additional hours, at least 32 additional hours, at least 40 additional hours; for cultured for 72 hours followed by: 8 additional hours, 16 additional hours, 24 additional hours, 32 additional hours, 40 additional hours, or 48 additional hours (i.e., up to 5 days total).

In the second stage of the method, which generates the PPCs, also referred to herein as “step (b)” or “stage 2”, the DFECs generated in step (a) are further cultured in the stage 2-optimized culture media on days 4-6, or starting on day 4 and continuing through day 6, or starting on day 4 and continuing for 72 hours (3 days), or starting on day 4 and continuing for at least 60 hours, or at least 64 hours, or at least 68 hours, or at least 70 hours, or at least 72 hours, or starting on day 4 and continuing for 60 hours, or for 64 hours, or for 68 hours, or for 70 hours, or for 72 hours. In some embodiments, the DFECs may be cultured for up to 5 days in stage 2 of the differentiation protocol. Therefore, in certain embodiments, the DFECs may be cultured in the stage 2-optimized culture media for 72 hours followed by: at least an additional 8 hours, at least an additional 16 hours, at least an additional 24 hours, at least an additional 32 hours, at least an additional 40 hours; or cultured for 72 hours followed by: 8 additional hours, 16 additional hours, 24 additional hours, 32 additional hours, 40 additional hours, or an additional 48 hours (i.e., up to 5 days total).

In the third stage of the method, which generates the PBCs, also referred to herein as “step (c)” or “stage 3”, the PPCs generated in step (b) are further cultured in the stage 3-optimized culture media on days 7-16, or starting on day 7 and continuing through day 16, or starting on day 7 and continuing for 240 hours (10 days), or starting on day 7 and continuing for at least 216 hours, or starting on day 7 and continuing for at least 220 hours, or starting on day 7 and continuing for at least 224 hours, or starting on day 7 and continuing for at least 228 hours, or starting on day 7 and continuing for 216 hours, or for 220 hours, or for 224 hours or for 228, or for 232 hours. In some embodiments, the PPCs may be cultured for up to 20 days in stage 3 of the differentiation protocol. Therefore, in certain embodiments, the PPCs may be cultured in the stage 3-optimized culture media for 240 hours (10 days) followed by: at least 1 additional day, at least 2 additional days, at least 3 additional days, at least 4 additional days, at least 5 additional days, at least 6 additional days, at least 7 additional days, at least 8 additional days, at least 9 additional days, or for 240 hours (10 days) followed by: 1 additional day, 2 additional days, 3 additional days, 4 additional days, 5 additional days, 6 additional days, 7 additional days, 8 additional days, 9 additional days, or 10 additional days (i.e., up to 20 days total).

In an exemplary embodiment, TC plates are seeded in Essential 8 (E8) media at an initial cell density of 62,500 cells/cm² and grown overnight. The following day, growth media is replaced with a stage 1 media composed of a basal media (CDM2) supplemented with 250 nM LDN193189, 2 μM retinoic acid, 500 nM A8301 and 250 nM PD0325901. The CDM2 media can be used in all stages of the differential protocol and has been described by Loh et al 2014. The culture is maintained in a stage 1 media for 3 consecutive days. On the third day the differentiation media is changed to a stage 2 media composed of the basal media CDM2 supplemented with 250 nM LDN193189, 500 nM (5Z)-7-Oxozeanol, 2 μM retinoic acid, 300 nM SUN 11602, 250 nM AT7867 and 500 nM A8301. Cultures are incubated in the stage 2 media for three consecutive days until the media is changed to a stage 3 media. The stage 3 media is composed of CDM2 supplemented with 100 nM gamma secretase, 500 nM A8301 and 15 μM quercetin. Cultures are incubated in the presence of the stage 3 media for 5-10 consecutive days.

The foregoing differentiation protocol has been shown to be highly reproducible and results in consistent differentiation regardless of the adherent system used and has been successfully employed in most TC format including 96-well, 24-well, 6-well and T75 flasks. This protocol has also been shown to function in 3D suspension cultures as described below. Throughout all stages, the differentiated PSC culture is provided fresh media daily through a media change.

In some cell suspension embodiments, a shaker flask expansion method may be employed, which consists of filling a flask approximately ⅓ of the way with growth media, adding PSCs, and placing the entire flask on a shaker so that PSCs do not settle and stay in suspension. The media solution is shaken so that the liquid is constantly circulating without splashing. This method, originally developed for bacterial or yeast growth, has been adapted for mammalian cell growth and subsequently for PSC aggregate growth. In other cell suspension embodiments, spinner flasks may be employed. Spinner flasks have a propeller attached to a rod that reaches down into the flask for stirring. The propellers are horizontal and are designed to be driven by magnetic stir plates that the flasks sit on.

In some embodiments, a bioreactor system is used for culturing the cells. An exemplary bioreactor system is the PBS Vertical Wheel Bioreactor system (https://www.pbsbiotech.com/uploads/1/7/9/9/17996975/2021_borys_et_al._-_overcoming_bioprocess_bottlenecks.pdf). The PBS VW Bioreactors include an enclosed vesicle that can accommodate suspension cultures of various sizes, including 100 ml, 500 ml, 3,000 ml, and 15,000 ml. The culture vessels have a corresponding motorized unit that they sit on and spin the magnetized vertical wheel allowing for constant stirring of the suspension culture. In one embodiment, StemScale media is used for the general expansion of PSCs within the bioreactor.

In an exemplary embodiment, the initial seeding into the VW Bioreactors comes from an adherent culture, though subsequent passaging events can be achieved from bioreactor to bioreactor. Multiple wells of a 3-4 day 6-well plate are washed with PBS followed by a 3-minute incubation in the presence of TryPLE to remove cells from the plate. Three to four of these wells are then washed with a basal media to remove any cells that are not completely de-attached from the plate. Cells are counted and are seeded at a concentration of 150,000 cells per ml into a PBS VW Bioreactor in StemScale media supplemented with 10 μM Y27632. The following day, and every 2^{nd} day after that, a demi-depletion is performed on the bioreactor. The demi-depletion is achieved by removing the bioreactor from its base and setting it in a hood for 5 minutes allowing aggregates to settle to the bottom of the bioreactor at which time half of the volume of the bioreactor is removed without disturbing the bottom of the reactor. This volume is then replaced with fresh Stem Scale media. Bioreactors are passaged every 4-5 days. All media containing aggregates are removed from the bioreactor and placed into tubes for centrifugation. Samples are spun at 400×g for 4 minutes followed by aspiration of the supernatant media leaving pellets of the cellular aggregates. This pellet is then incubated in the presence of accutase for 10 minutes to break the aggregates up into smaller pieces and individual cells. The accutase is then diluted centrifuged so that it can be aspirated out of the sample. Passaging within the bioreactor content can be done in a scale up model or in a continuous culture model. Scale up occurs when all biomaterial is placed into a larger bioreactor for subculturing, such as the next size bioreactor for passaging a 100 ml sample into a 500 ml bioreactor, etc. The continuous model may be employed when only a portion of the sample is sub-cultured in a subsequent bioreactor, such as when the sample is processed from a 100 ml run and only a portion of it is then seeded back into a 100 ml bioreactor.

### IV. Uses

The methods and compositions of the disclosure for generating DFECs, PPCs, and PBCs allow for efficient and robust availability of these cell populations for a variety of uses in clinical therapy, research, development, and commercial purposes. For therapeutic purposes, the in vitro generated PBCs of the present invention can be administered systemically or directly to a subject for treating or preventing type 1 diabetes, type 2 diabetes, pre-diabetes, conditions due to significant trauma (i.e., damage to the pancreas or loss or damage to islet beta cells), or treating other metabolic diseases or disorders associated with a deficiency in beta cell number (e.g., a reduction in the number of pancreatic cells), an insufficient level of beta cell biological activity (e.g., a deficiency in glucose-stimulated insulin secretion, a deficiency in insulin production).

In one embodiment, the PBC cells of the invention are directly injected into an organ of interest (e.g., pancreas). Alternatively, compositions comprising beta-like cells of the invention are provided indirectly to the organ of interest, for example, by administration into the circulatory system (e.g., the pancreatic vasculature). Expansion and differentiation agents can be provided prior to, during or after administration of the cells to increase production of cells having insulin-producing potential in vitro or in vivo. In some embodiments, the PBCs of the present application may be genetically modified to increase their therapeutic and/or safety profiles prior to administration. The cells can be administered in any physiologically acceptable vehicle, normally intravascularly, although they may also be introduced into another convenient site where the cells may find an appropriate site for regeneration and differentiation.

In one embodiment, at least 100,000, 250,000, or 500,000 cells are injected. In other embodiments, 750,000, or 1,000,000 cells are injected. In other embodiments, at least 1×10⁵ cells, at least about 1×10⁶, at least about 1×10⁷, or as many as 1×10⁸ to 1×10¹⁰, or more are administered. Dosages can be readily adjusted by those skilled in the art (e.g., a decrease in purity may require an increase in dosage). The PBCs cells can be introduced by localized injection, including catheter administration, systemic injection, intravenous injection, or parenteral administration. When administering a therapeutic composition of the present invention (e.g., a pharmaceutical composition containing a selected cell), it will generally be formulated in a unit dosage injectable form (solution, suspension, emulsion).

In other embodiments, the PBCs cells of the disclosure can be used for screening potential drugs or for the development of novel cell therapies for treatment of diseases or disorders involving dysfunction of PBCs.

In other embodiments, the methods and compositions can be used in the study of pancreatic beta cell progenitor development and biology, including differentiation into PBCs, to assist in the understanding and potential treatment of diseases and disorders associated with abnormal pancreatic beta cell function, such as diabetes.

In certain embodiments, DFECs, PPCs, and PBCs generated using the methods of the disclosure can be further purified according to methods established in the art using agents that bind to surface markers expressed on the cells. Accordingly, in one embodiment, the disclosure provides a method of isolating DFECs, PPCs, and PBCs, the method comprising: contacting DFECs, PPCs, or PBCs generated by a method of the disclosure with one or more binding agent(s) (e.g., monoclonal antibodies (mAbs)) binding to one or more cell surface marker(s) expressed in DFECs, PPCs, or PBCs; and isolating cells that bind to the binding (s) to facilitate isolation of the DFECs, PPCs, or PBCs. Cells that bind the antibody can be isolated by methods known in the art, including but not limited to fluorescent activated cell-sorting (FACS) and magnetic activated cell sorting (MACS).

### V. Compositions

In other aspects, the disclosure provides compositions related to the methods of generating DFECs, PPCs, and PBCs, including culture media and cell cultures, as well as isolated progenitor cells and cell populations thereof.

In one aspect, the disclosure provides a culture media for obtaining human DFEs comprising culture media comprising a BMP pathway antagonist, a RA pathway agonist, a TGF-β pathway antagonist, and an MEK pathway antagonist. Suitable agents, and concentrations therefor, include those described in subsection II.

In another aspect, the disclosure provides a culture media for obtaining human PPCs comprising a BMP pathway antagonist, a RA pathway agonist, a TGF-β pathway antagonist, a TAK1 pathway antagonist, a bFGF mimetic, and an Akt pathway antagonist. Suitable agents, and concentrations therefor, include those described in subsection II.

In another aspect, the disclosure provides culture media for obtaining human pancreatic beta cells (PBCs) comprising a Notch pathway antagonist, such as a γ-secretase inhibitor, a TGF-β pathway antagonist, and a flavonoid, such as quercetin or a quercetin analogue. Suitable agents, and concentrations therefor, include those described in subsection II.

In another aspect, the disclosure provides an isolated cell culture of human DFECs, the culture comprising human DFECs cultured in a culture media comprising a BMP pathway antagonist, a RA pathway agonist, a TGF-β pathway antagonist, and an MEK pathway antagonist. Suitable agents, and concentrations therefor, include those described in subsection II.

In another aspect, the disclosure provides an isolated cell culture of human PPCs, the culture comprising human PPCs cultured in a culture media comprising a BMP pathway antagonist, a RA pathway agonist, a TGF-β pathway antagonist, a TAK1 pathway antagonist, a bFGF mimetic, and an Akt pathway antagonist. Suitable agents, and concentrations therefor, include those described in subsection II.

In another aspect, the disclosure provides an isolated cell culture of human pancreatic beta cells (PBCs) the culture comprising human PBCs cultured in a culture media comprising a Notch pathway antagonist, such as a γ-secretase inhibitor, a TGF-β pathway antagonist, and flavonoid, such as quercetin or a quercetin analogue. Suitable agents, and concentrations therefor, include those described in subsection II.

In another aspect, the disclosure provides human DFECs generated by a method of the disclosure (i.e., step (a) or stage 1 of the culture protocol).

In another aspect, the disclosure provides human PPCs generated by a method of the disclosure (i.e., steps (a) and (b), or stages 1 and 2 of the culture protocol).

In another aspect, the disclosure provides human PBCs generated by a method of the disclosure (i.e., steps (a), (b) and (c), or stages 1, 2 and 3, of the culture protocol).

The present invention is further illustrated by the following examples, which should not be construed as further limiting. The contents of figures and all references, patents and published patent applications cited throughout this application are expressly incorporated herein by reference.

### EXAMPLES

### Example 1 & 2: Optimization of HNF1B from the IPSC-line CR01

The Dorsal Foregut Endoderm (DFE) is defined as a pluripotent derived endodermal population patterned to a region of the early developing embryo occurring on the dorsal side of the midgut of the primitive gut-tube. Expression patterns within this population are marked by robust expression of the endodermal dorsal markers of SFRP5, MNX1, PTCH1 and PAX6 and the midgut markers FOXA2, HNF1b, HOXA3 and ONECUT2. The high expression of these markers contrasts with the absence of these markers of the ventral and more posterior endodermal regions. DFE was found to exhibit very low or no expression of the ventral markers HHEX and NR5A2, as well as the classically defined definitive endodermal markers SOX17, GSC, MIXL1 and CER. It was previously demonstrated that the DFE state could be achieved through the dual optimization of HNF1b and FOXA2 and that this induction could be achieved through the retinoid signaling occurring in the absence of BMP signaling. Previous work performed on the DFE population used a single iPSC line and embryonic stem cells.

To assay the efficacy of a potentially clinical grade iPSC line the NCRM1 cell line was chosen. An HD-DoE based optimization was used to validate the HNF1b and FOXA2 optimizers for this cell line. The HD-DOE method was applied with the intent to find conditions for induction of endoderm-expressed genes, directly from the pluripotent stem cell state. This example utilizes a method previously described by Bukys et al. (2020) Iscience 23:101346. The method employs computerized design geometries to simultaneously test multiple process inputs and offers mathematical modeling of a deep effector/response space. The method allows for finding combinatorial signaling inputs that control a complex process, such as dauring cell differentiation. It allows testing of multiple plausible critical process parameters, as such parameters impact output responses, such as gene expression. Because gene expression provides hallmark features of the phenotype of, for example, a human cell, the method can be applied to identify, and understand, which signaling pathways control cell fate.

To develop a cell culture recipe for each stage, the impact of agonists and antagonists of multiple signaling pathways (herein called effectors) on the expression of pre-selected genes was tested and modeled. The impact of each effector on gene expression level is defined by a parameter called factor contribution that is calculated for each effector during the modeling. These effectors are small molecules or proteins that are commonly used during stepwise differentiation of stem cells to specific fates. Choice of the effectors were based on current literature on differentiation of stem cells to endoderm progenitors.

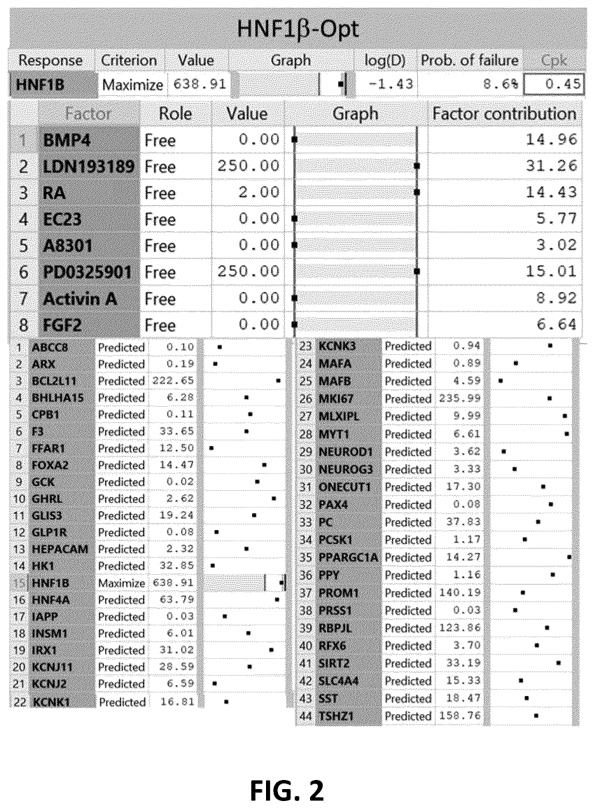

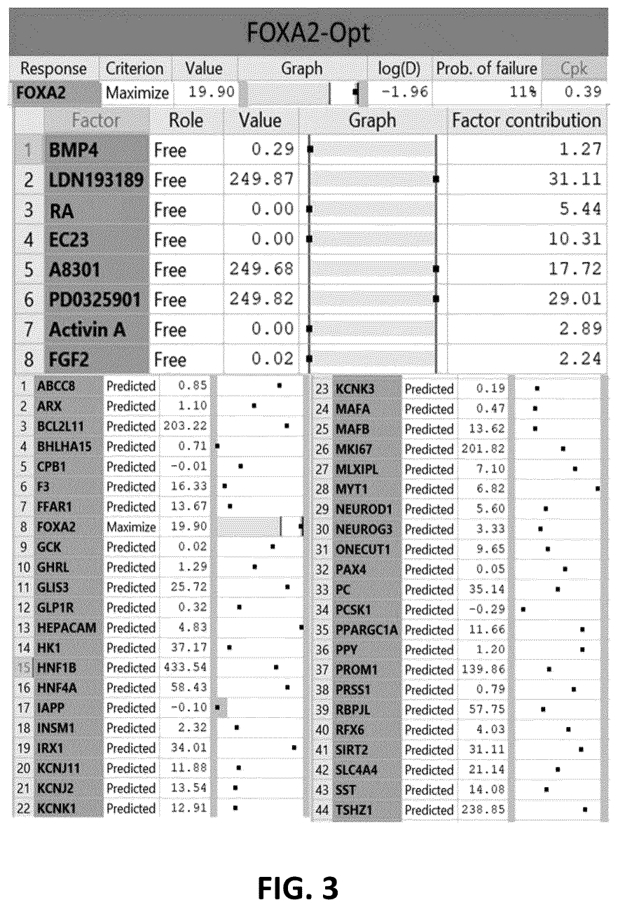

In both optimizations, it was shown that BMP pathway inhibition was the most crucial component of the optimization with factor contribution factors of 31.26 and 31.11 for LDN193189 in both the optimization of HNF1b (FIG. 2) and FOXA2 (FIG. 3), respectively. While MEK pathway inhibition with PD0325901 was critical for both HNF1b and FOXA2 optimization (factor contributions of 15.01 and 29.01, respectively), FOXA2 activation was more dependent on MEK inhibition. The other two pathways that were determined to be crucial for controlling DFE activation were retinoid signaling and TGF-β pathway inhibition. These two pathways had differential effects on FOXA2 and HNF1b. Inhibition of TGF-β pathway using A8301 was crucial for optimal FOXA2 activation (FIG. 3) with a positive correlation and a significant FC of 17.72, while the use of A8301 had little effect on HNF1b activation (FIG. 2, negative correlation with a negligible FC of 3.02). Retinoid signaling was significant in the activation of HNF1b with a FC of 14.43 (FIG. 2), but not significantly involved in FOXA2 activation with a negative correlation and a FC of 5.44 (FIG. 3).

### Example 3: Dual Optimization of FOXA2 and HNF1B Generates Dorsal Foregut Endoderm (DFE)

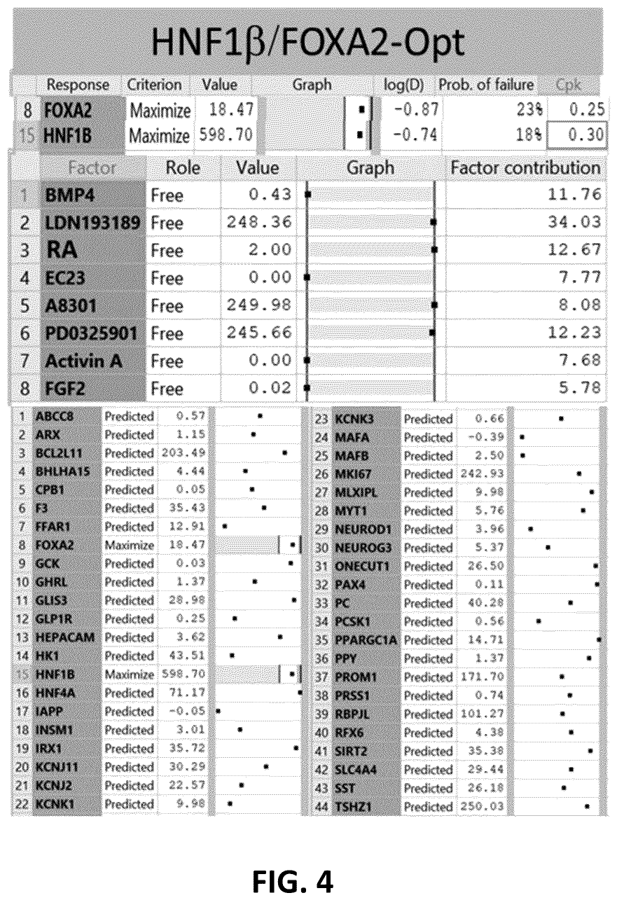

Performing a dual optimizer for the combined expression of FOXA2 and HNF1b further demonstrated the importance of simultaneously controlling these four pathways as the optimizer predicted that active retinoid signaling was needed in the presence of BMP, TGF-β and MEK pathway inhibition (FIG. 4). It was concluded that while both retinoid signaling and BMP pathway inhibition are still needed for DFE generation, the CR01 iPSC cell line has an increased need for both the active inhibition of TGF-β and MEK signaling.

### Example 4: Dynamic Profiling Demonstrates the Requirement for MEK and TGF-β Inhibition for FOXA2 Induction

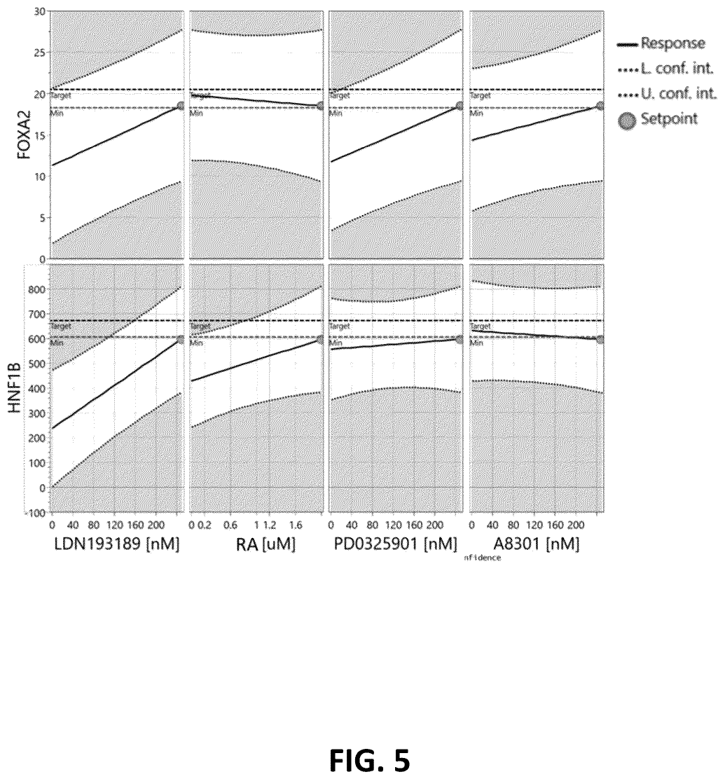

As shown in FIG. 5, dynamic profiling further confirmed the need to control all four pathways simultaneously. The most critical process parameter to control in the generation of DFE generation is the inhibition of BMP signaling by LDN193189. This was crucial for both HNF1b and FOXA2 expression, whereas the other components present in the HNF1B/FOXA2 optimizer had differential effects on these two fate-driving genes. Retinoid signaling was shown to greatly increase HNF1b activation while having a slightly inhibitory effect on FOXA2 expression, though this inhibitory effect was negligible. Similarly, the inhibition of both the MEK and TGF-β pathways were highly beneficial for the activation of FOXA2 while having little to no effect on HNF1b expression.

### Example 5: Coefficient Plots Show Main Effectors Needed for DFE Differentiation

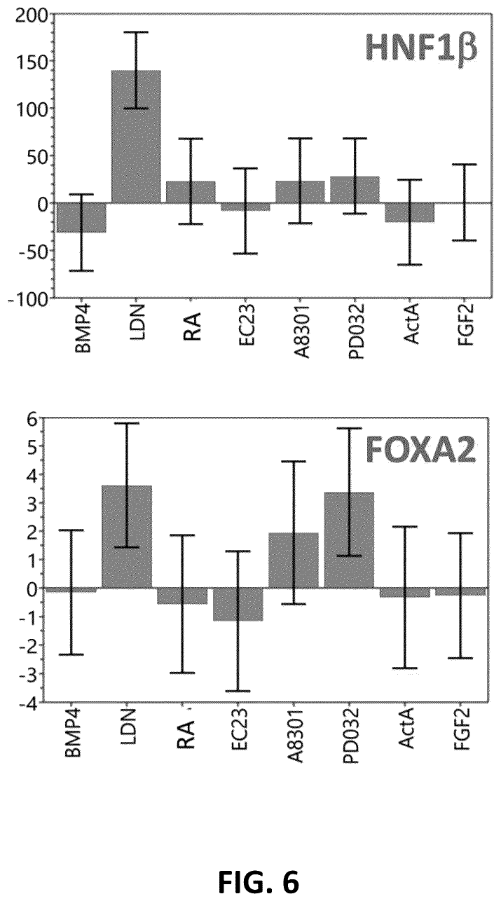

As shown in FIG. 6, coefficient plots confirmed the fundamental importance of BMP pathway inhibition in the activation of both HNF1b (top portion) and FOXA2 (bottom portion). Inhibition of the TGF-β and MEK pathways was determined to be more directly involved in the activation of FOXA2.

### Example 6: Heat Map Confirms the Critical Input Components Needed for DFE Generation

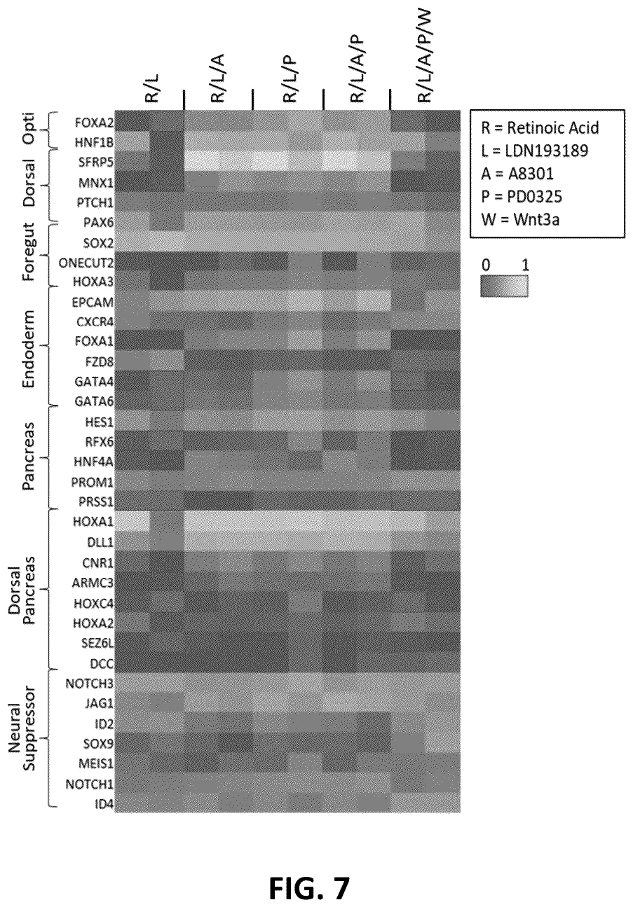

Use of the previously identified critical process parameters retinoic acid and LDN193189 in the presence or absence of A8301 and PD0325901 showed the dependence of robust FOXA2 and HNF1b on the addition of either of these compounds (FIG. 7). The provision of active Wnt signaling (HNF1b optimizer identified on embryonic stem cells) to this differentiation media split the co-expression of HNF1b from that of FOXA2 and greatly reduced endodermal genes overall suggesting a difference between the pluripotent starting material. By actively inhibiting both the TGF-β and MEK pathways, increases in genes representative of a dorsal, foregut and generalized endodermal fate were observed (FIG. 7). In addition, a greater pancreatic fate was observed in this progenitor population. All conditions shown demonstrated genes known to suppress neural fate.

### Example 7: DFE is Directly Induced without Going Through a Primitive Streak or a Definitive Endoderm Intermediate

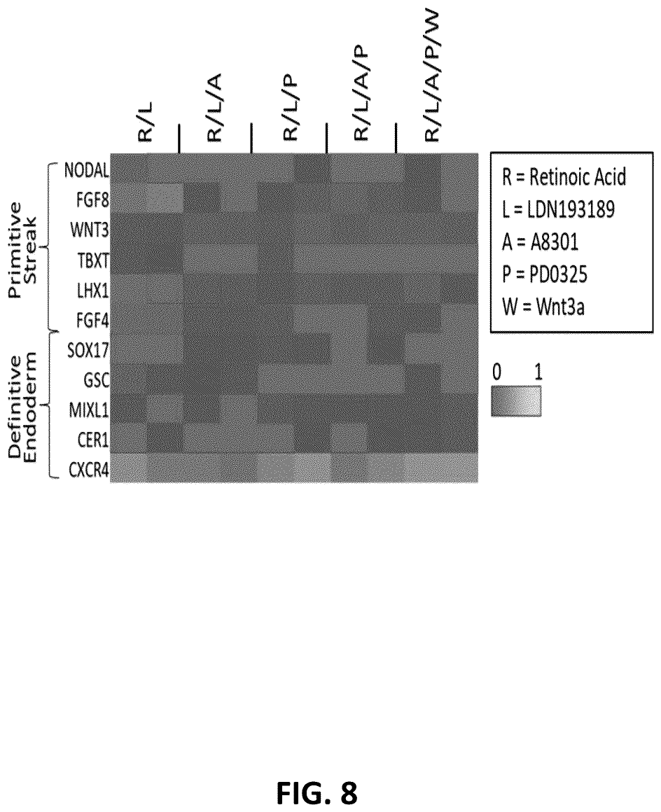

As shown in the heatmap depicted in FIG. 8, genes classically defined as primitive streak and definitive endoderm markers were not activated during the induction of DFE further demonstrating the direct conversion of iPSC to a patterned endodermal fate. None of the genes classically defined as primitive streak marker showed any level of activation including the NODAL, FGF8, WNT3, TBXT, LHX1 and FGF4. Similarly, the definitive endodermal markers SOX17, GSC, MIXL1 and CER1 were not expressed, except the DE markers associated with a DFE patterning, FOXA2 and CXCR4.

### Example 8: Achieving Optimal FOXA2 Expression

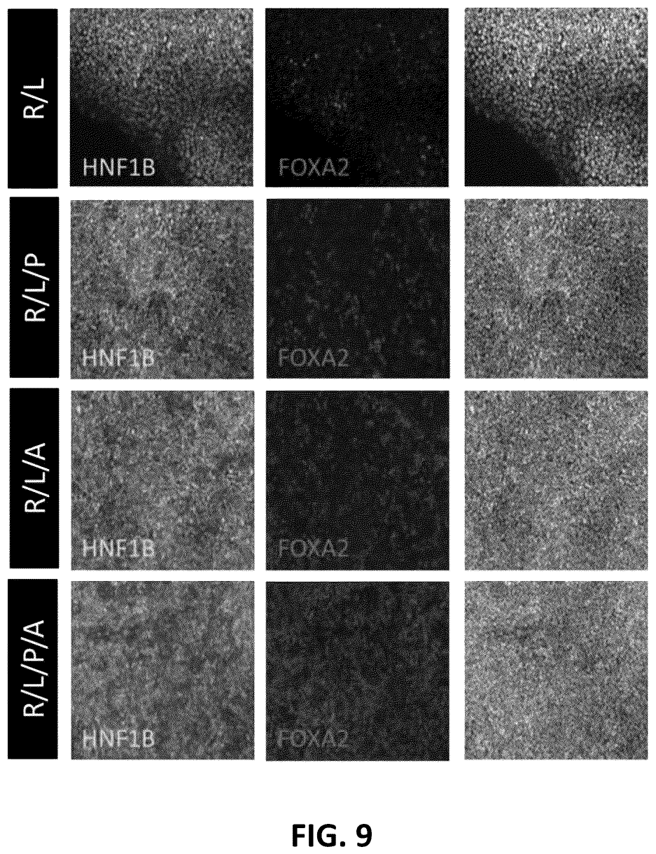

As shown in FIG. 9, immunohistochemical analysis confirmed the increased contribution of MEK (PD0325901) and TGF-β (A8301) pathway inhibition on the activation of FOXA2. Specifically, inclusion of either PD0325901 or A8301 increased FOXA2 activation over RA and LDN alone, however this increase was compounded when both PD0325 and A8301 were used together.

### Example 9: Dynamic Profiling Demonstrates Pancreatic Potential

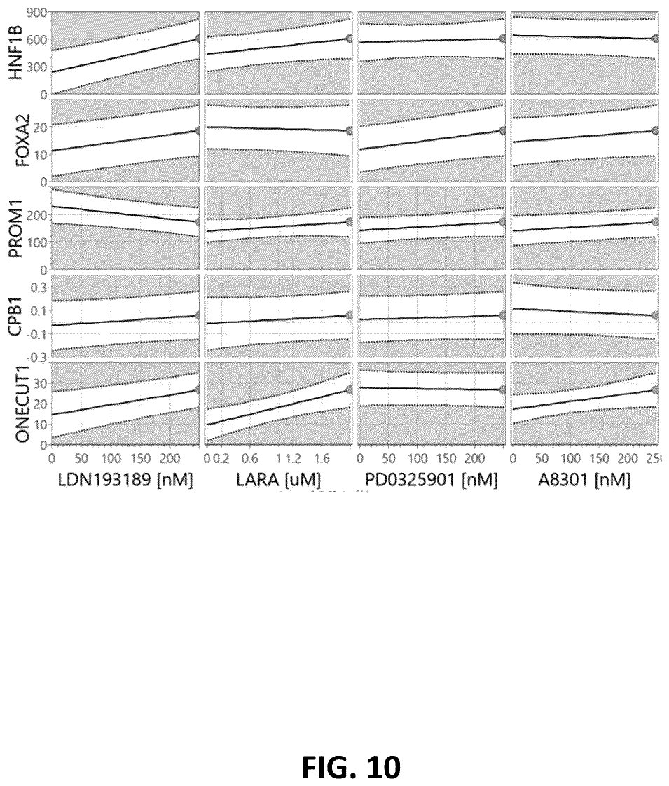

To assay the capacity to generate pancreatic field from the DFE culture, genes associated with this endodermal region were evaluated for expression in pancreatic bud generation. These genes include PROM1, CPB1 and ONECUT1. As shown in the dynamic profiling depicted in FIG. 10, these genes were shown to be regulated in a similar manner and were all being activated within the DFE cultures. The combined effect of BMP pathway inhibition and active retinoid signaling increased activation of both ONECUT1 and CPB1. While PROM1 activation did not rely on BMP inhibition, it exhibited a stronger response to the combined effects of retinoid signaling in the presence of TGF-β and MEK inhibition. Altogether, the combined expression of HNF1B, FOXA2, PROM1, CPB1 and ONECUT1 indicates an endodermal population capable of pancreatic field induction.

### Example 10: Modelling for Maximal PDX1 Induction

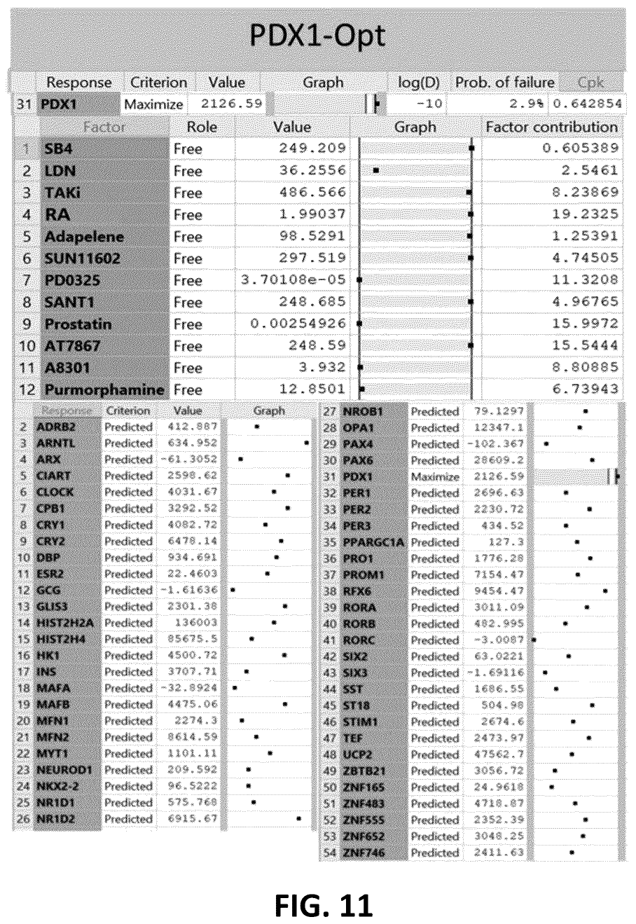

To determine the best way for inducing a pancreatic fate from a DFE progenitor, a subsequent HD-DoE analysis was performed. Exposing a DFE culture to a perturbation matrix composed of all previously identified effectors shown to have a positive effect on PDX1 activation was used to define optimal conditions for PDX1 induction. As shown in FIG. 11, retinoid signaling (retinoic acid) and inhibition of the Akt/PKA pathways (AT7867) had the strongest effects on PDX1 activation with contribution factors of 19.2 and 15.5 respectively. In addition, inhibition of the TAK1 pathway ((5Z)-7-oxozeanol) and the SHH pathway (SANT1) while stimulating the FGF pathway (SUN11602) had decreasing contributions to the activation of PDX1 with factor contributions of 8.2, 5.0 and 4.7 respectively.

### Example 11: Coefficient Plots Show Main Effectors for Pancreatic Progenitor Generation from a DFE Precursor

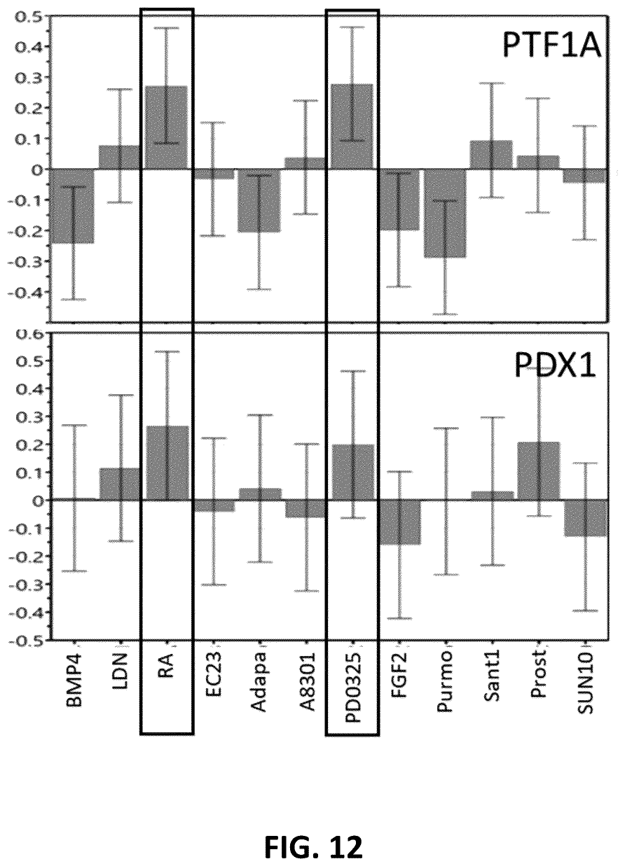

Coefficient plots comparing the input logic of the pancreas specific genes PTF1A and PDX1 showed that the combined effects of active retinoid signaling (retinoic acid) in the presence of MEK pathway inhibition (PD0325901) are the two pathways most critical to control for the activation of the pancreatic field from a regionalized dorsal foregut endodermal field (FIG. 12). The combined expression of PTF1A and PDX1 only occurs within the early pancreatic field of the developing embryo and does not occur in any other tissue type.

### Example 12: Optimizing for PDX1 Induction

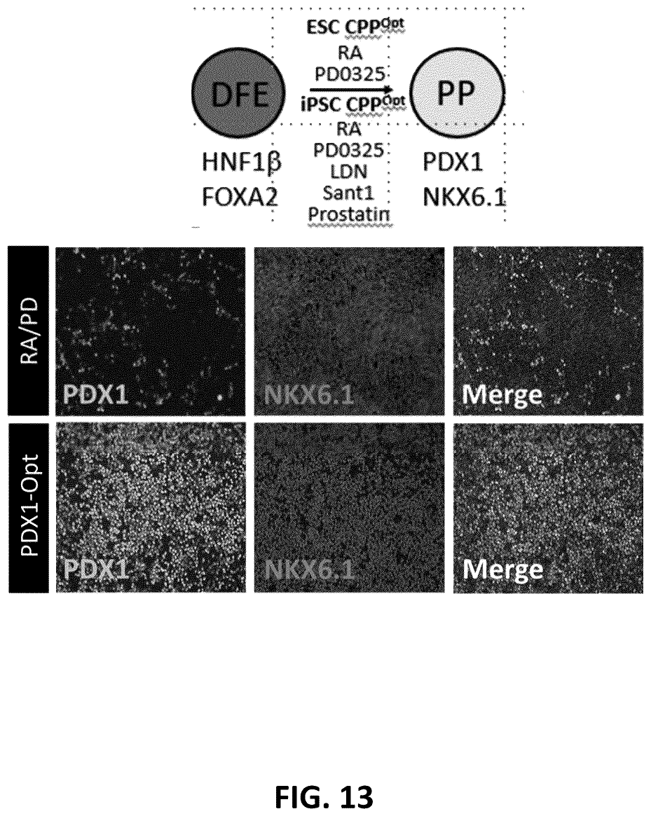

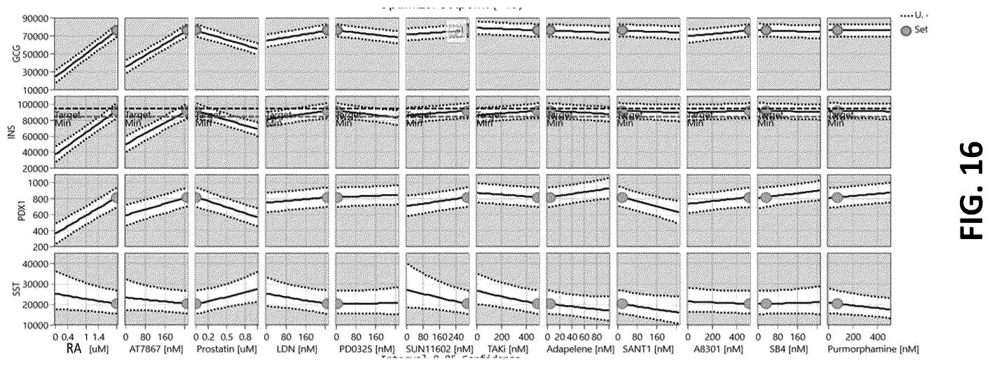

As shown in FIG. 13, immunohistochemical analysis demonstrated that even though the main factors needed for PDX1 activation are retinoic acid and PD0325901, inclusion of the other indicated pathways greatly increased PDX1 activation also. Inhibition of both the BMP (LDN) and SHH (sant1) pathways, while providing PKC agonism, increased PDX1 activation as shown in FIG. 16 of Example 15.

### Example 13: Dynamic Profiling Exploring Endocrine Cell Sub-Types

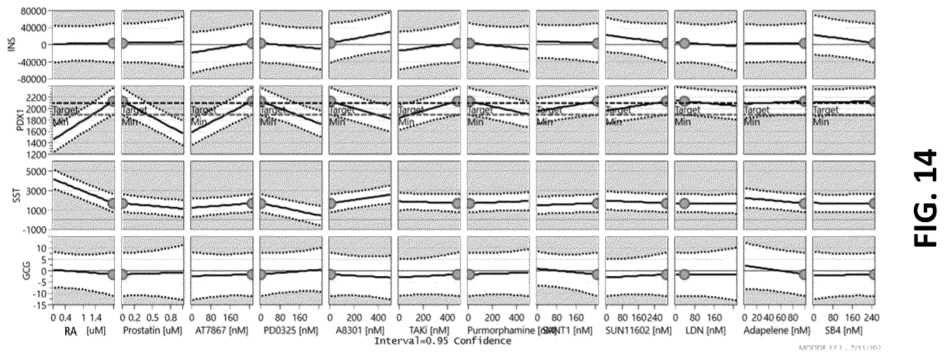

Comparing the PDX1 optimizer to markers of the pancreatic endocrine fates showed that the predicted conditions were compatible with the generation of endocrine fates (FIG. 14). Notable only retinoic acid had a strong inhibitory effect on the activation of SST (delta cell marker) and the FGF agonist SUN11602 had a slightly negative effect on INS (beta cell marker) activation, thereby suggesting that this predicted PDX1 induction was representative of a proendocrine field.

### Example 14: Modelling for Maximal Insulin Expression

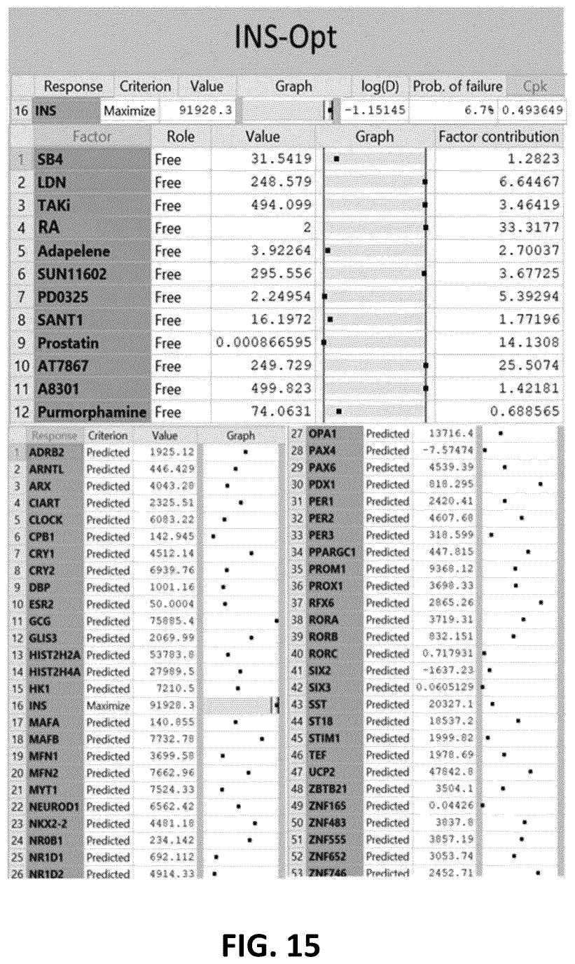

Since PDX1 positive cells can give rise to all cell types of the pancreas and gall bladder, as well as cells of the stomach and intestines, optimal conditions for beta cell induction were explored. This was accomplished through a parallel experiment exposed to the same perturbation matrix that was differentiated an additional 5 days afterwards in the presence of both a Notch pathway inhibitor (gamma secretase inhibitor (GSI)-XX) and a TGF-β pathway inhibitor (A8301). It is well established that inhibition of these pathways induces a beta cell fate from a proendocrine progenitor field. Optimization of this culture for the INS predicted a similar culture condition as the original PDX1 optimizer. Notably, as shown in FIG. 15, both contribution factors for the retinoid agonist and the Akt/PKA pathway inhibitor increased with factor contributions of 33.3 and 25.5 respectively. In addition, while there was an increased need for the inhibition of both the BMP pathway (LDN) and the TGF-β pathway (A8301), active inhibition of the SHH pathway was no longer needed.

### Example 15: Dynamic Profiling of Endocrine Cell Sub-Types

Comparing the regulatory inputs of the effectors used in the insulin optimizer to PDX1 activation showed almost identical input logic for the two genes. As shown in FIG. 16, the strongest regulatory inputs for the activation of both PDX1 and INS are active retinoid signaling and inhibition of Akt/PKA pathway. While GCG (alpha cell marker) showed an identical input logic to that of INS, SST (delta cell marker) displayed an input logic that was opposite of PDX1/INS/GCG, suggesting a highly different regulatory system for the delta cell.

### Example 16: Immunohistochemical Validation of De Novo Beta Cells

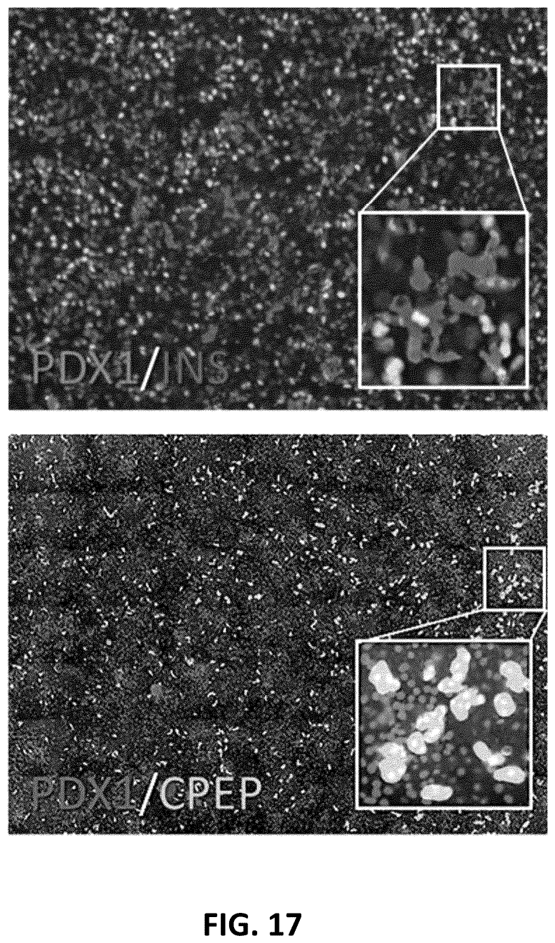

As shown in FIG. 17, immunohistochemical analysis confirmed the ability to activate endocrine genes from a PDX1 optimized culture. PDX1 expressing cultures were subjected to the TGF-β pathway inhibitor A8301 and the NOTCH pathway inhibitor gamma secretase inhibitor XX (GSI-XX) and subsequently assayed for INS expression. The results of this analysis demonstrated that the PDX1 field that was generated was proendocrine. Further, C-peptide staining confirmed that the INS detected was generated within the culture and was not absorbed from the growth media used to maintain the culture (FIG. 17). C-peptide is produced when proinsulin is processed to a functioning insulin molecule and is only present when de novo insulin is produced.

### Example 17: HD-DoE Screening for Optimal De Novo Beta Cell Induction

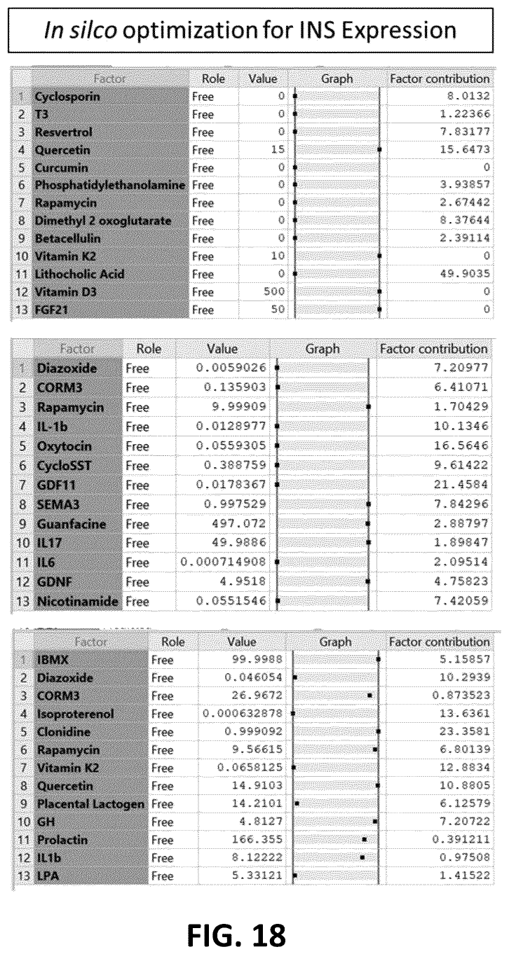

To define factors contributing to the activation of INS, a series of HD-DoE perturbations were performed using factors chosen to target different aspects of beta cell biology. As shown in FIG. 18, compounds exhibiting the greatest contribution to activation of INS targeted inhibition of the PI3K/AKT pathway (quercetin) and α2-adrenergic agonism (clonidine) with contribution factors of 15.6 and 23.4 respectively. Other compounds increased INS induction to a lesser extent, but with factor contributions ranging between 5-10 were SEMA3, GH and Rapamycin.

### Example 18: Heat Map Showing Descending Factor Contributions for the Induction of INS

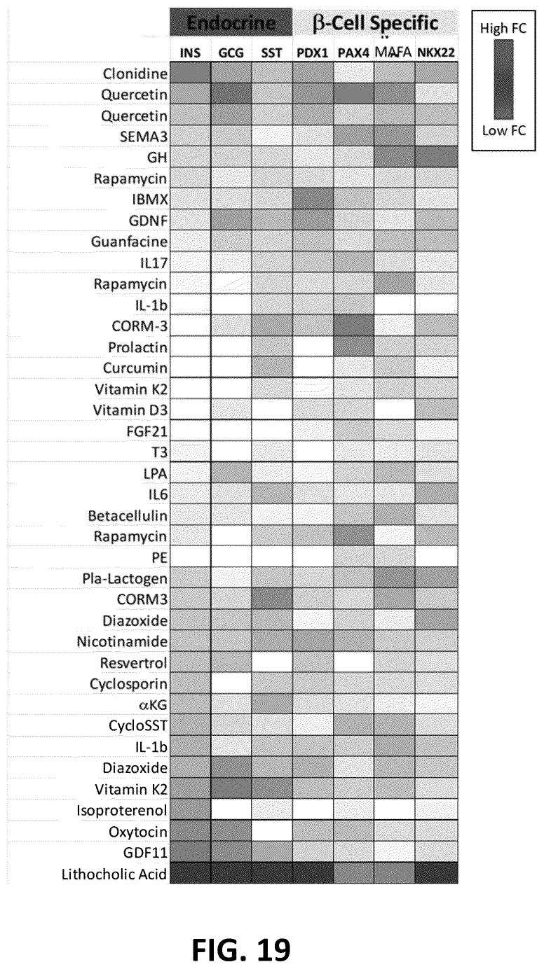

To ascertain which factors could potentially favor the beta cell over other endocrine cell types, optimizers for markers of the other endocrine cell types and beta cell specific markers were compared. The relative factor contribution from the different optimizers were compared. As shown in FIG. 19, compounds that were shown beneficial for all genes optimized in this fashion were the PI3K/AKT pathway inhibitor quercetin, the mTOR pathway inhibitor rapamycin, and the cAMP pathway activator IBMX.

### Example 19: HD-DoE Screening for Optimal De Novo Beta Cell Induction

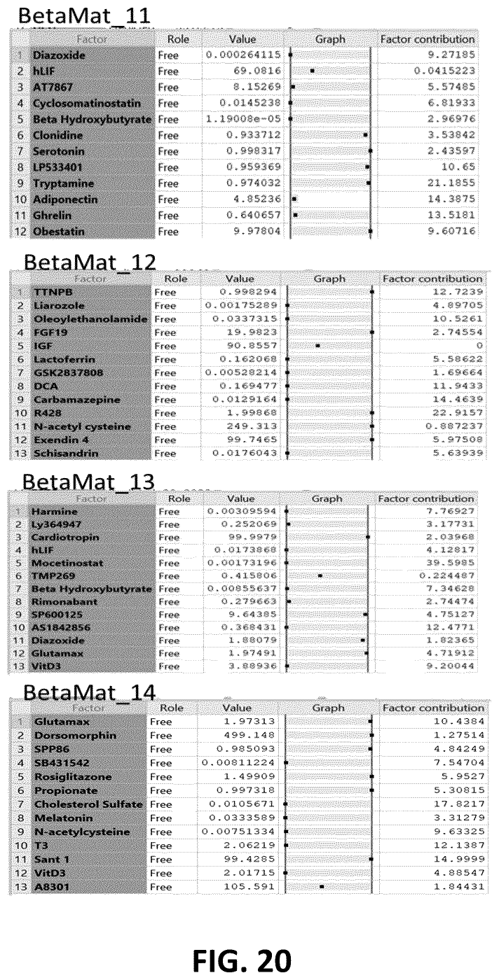

To define factors contributing to the activation of INS, a series HD-DoE perturbations were performed using factors chosen to target different aspects of beta cell biology. Of the factors tested, the ones determined to have strong INS activating capacity included the Ax1 inhibitor R248, the SHH inhibitor SANT1, the retinoid agonist TTNPB, the TPH1 inhibitor LP533401, and the amino acids tryptamine and glutamine (FIG. 20). Their respective factor contributions are 22.9, 15.0, 12.7, 10.7, 21.2, and 10.4, respectively. Other compounds that had a positive effect on the induction of INS included: the hormone obestatin, the GLP-1 activator exendrin 4, the RET pathway inhibitor SPP86, the thiazolidinedione rosiglitazone and the short chain fatty acid propionate. The contribution factors of these compounds for the induction of INS are 9.6, 6.0, 4.8, 6.0 and 5.3, respectively (FIG. 20).

### Example 20: Heat Map Showing Descending Factor Contributions for the Induction of INS

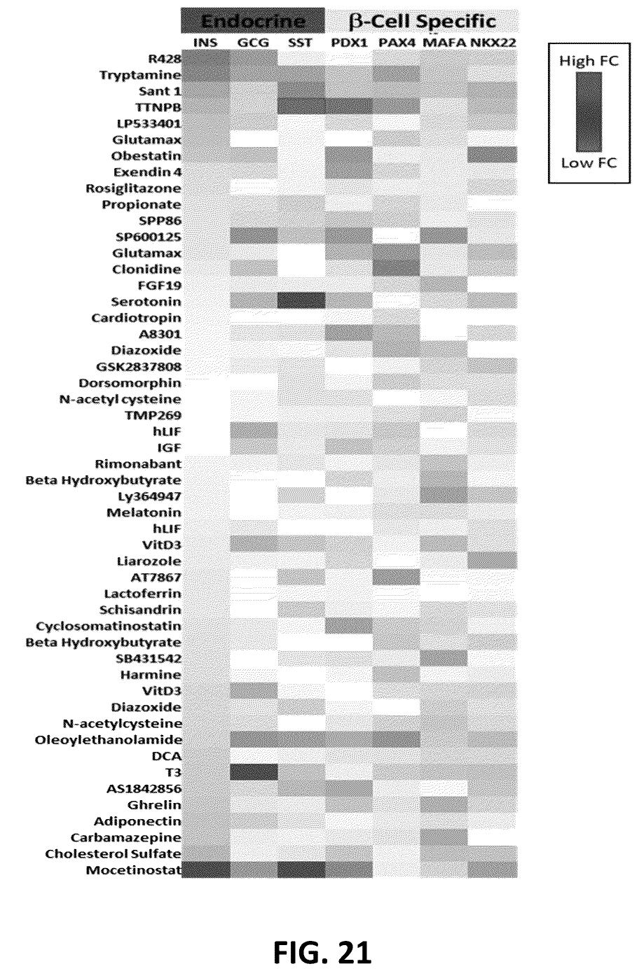

Optimizers for the main pancreatic endocrine hormones and beta cell-specific transcription factors were generated to compare their contribution factors for this series of HD-DoE. Compounds that could selectively favor the differentiation of the beta cell over that of the alpha and delta cells were identified in this manner. Notably, the retinoid agonist TTNPB was shown to strongly activate all beta cell specific transcription factors assayed in this manner while strongly inhibiting both the alpha and delta cell fates (FIG. 21). Propionate and glutamax had a similar effect of preferentially favoring the beta cell fate, though to a lesser level. While serotonin had little effect on INS induction, it strongly activated GCG induction, and inhibited SST induction (FIG. 21).

### Example 21: Dynamic Profiling of Endocrine Sub-Type Regulation

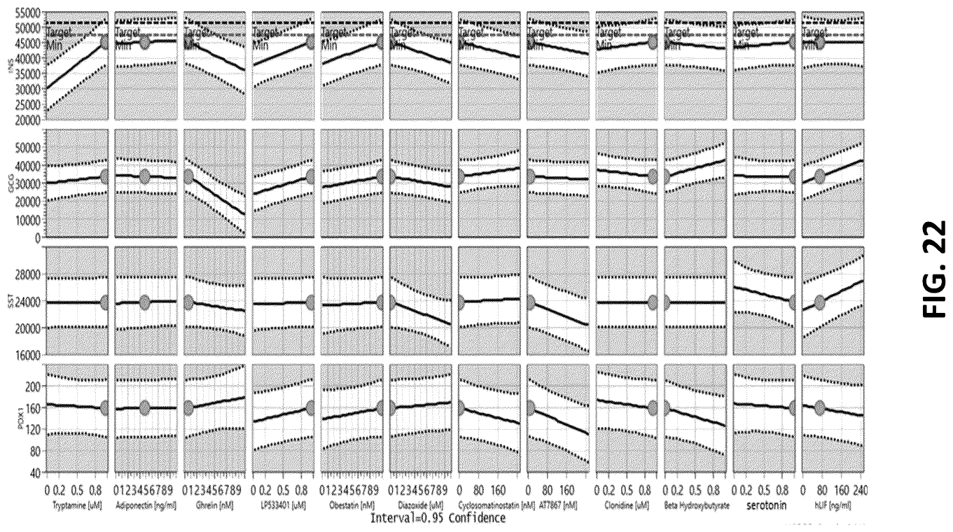

Dynamic profiling of the crucial beta cell specific markers INS and PDX1 was used to compare the regulatory profile of the beta cell to that of the alpha and delta cells (FIG. 22). Tryptamine had the strongest INS inducing effects within this HD-DoE matrix, selectively increasing INS induction while having no effect on any of the other transcripts measured. Other compounds that contributed to the induction of INS were LP533401, obestatin, clonidine and serotonin, though they had a lesser effect and were not specific for beta cell induction.

### Example 22: Dynamic Profiling of Endocrine Sub-Type Regulation

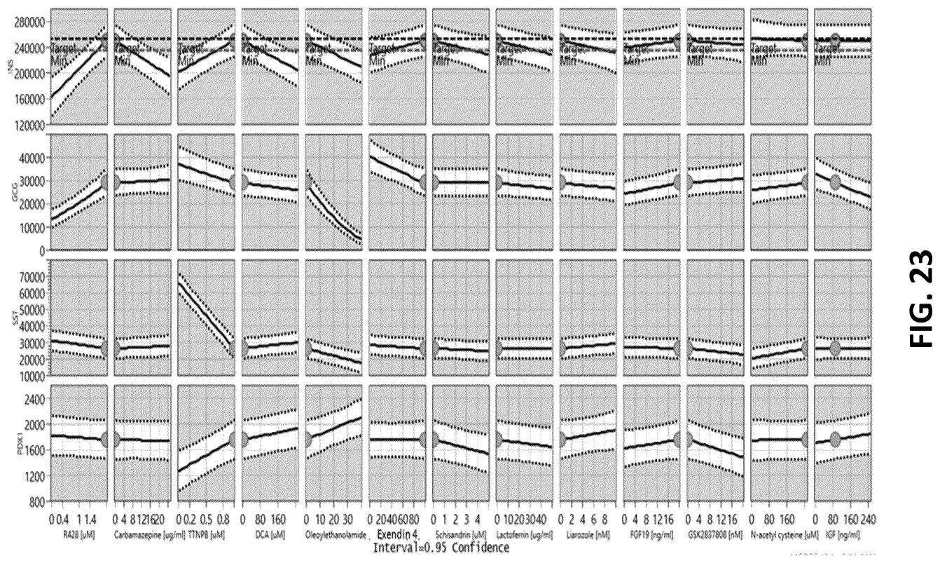

Dynamic profiling of the crucial beta cell specific markers INS and PDX1 was used to compare the regulatory profile of the beta cell to that of the alpha and delta cells (FIG. 23). The strongest inducers of the beta cell state were the Ax1 inhibitor R428 and the retinoid agonist TTNPB. TTNPB is shown to be highly selective for the beta cell state as it is inhibitory towards the divergent endocrine fates of the alpha and delta cells. Exendin 4 showed a similar pattern preferentially driving the beta cell fate from a proendocrine culture, though the induction of INS was not as strong with this compound. The FGF pathway agonist FGF19 also contributed to overall INS induction, but to a much smaller degree than the other mentioned effectors.

### Example 23: Dynamic Profiling of Endocrine Sub-Type Regulation

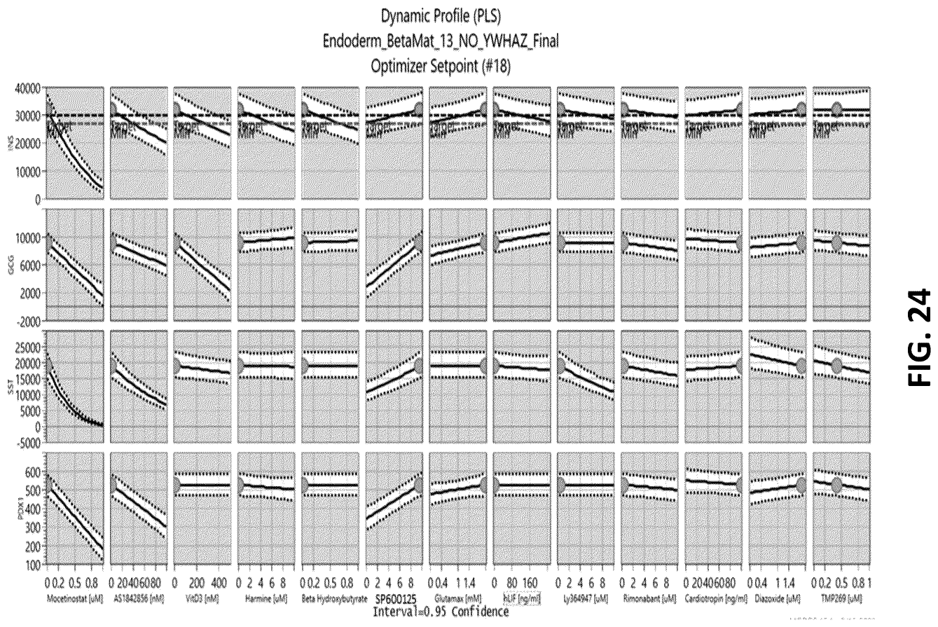

Dynamic profiling of the crucial beta cell specific markers INS and PDX1 was used to compare the regulatory profile of the beta cell to that of the alpha and delta cells. As shown in FIG. 24, the JNK pathway inhibitor SP600125 and the amino acid glutamax were both shown to be positive inducer of endocrine fate, however neither were beat cell specific as they also increased GCG and SST levels throughout the culture. Cardiotropin and diazoxide both positively increased INS induction, though not significantly.

### Example 24: Dynamic Profiling of Endocrine Sub-Type Regulation

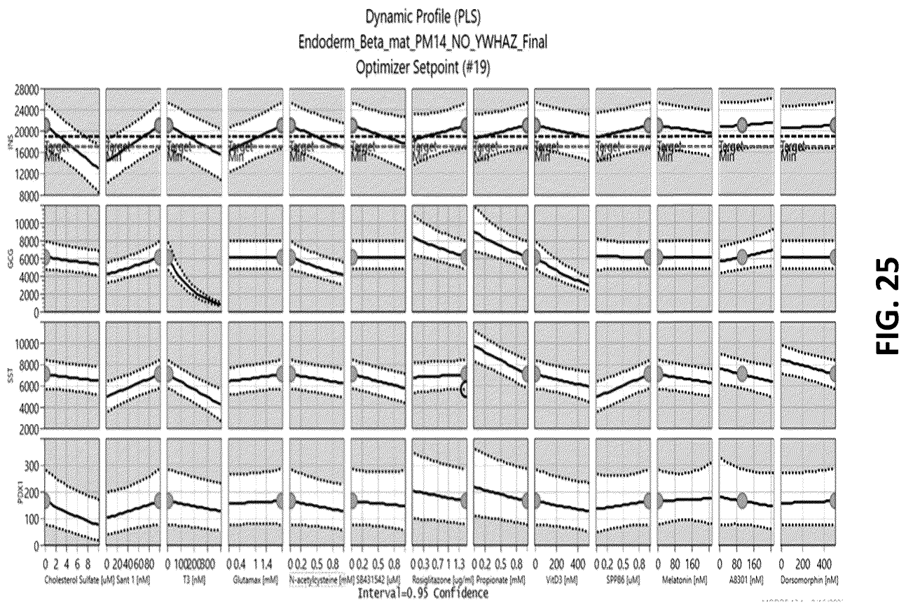

Dynamic profiling of the crucial beta cell specific markers INS and PDX1 was used to compare the regulatory profile of the beta cell to that of the alpha and delta cells. As shown in FIG. 25, the SHH pathway inhibitor SANT1 and glutamax both increased induction of endocrine fates without total specificity towards the beta cell. Though SANT1 and glutamax effects were stronger for INS induction, increased induction was observed for both GCG and SST. The compounds rosiglitazone and propionate significantly increased INS levels and inhibited both GCG and SST induction, suggesting specificity towards a beta cell state. Other compounds that had a positive effect on INS induction, though with far less contribution to the overall INS levels were SPP86, A8301 and dorsomorphin.

### Example 25: Immunohistochemical Validation of the Generation of the De Novo Beta Cells

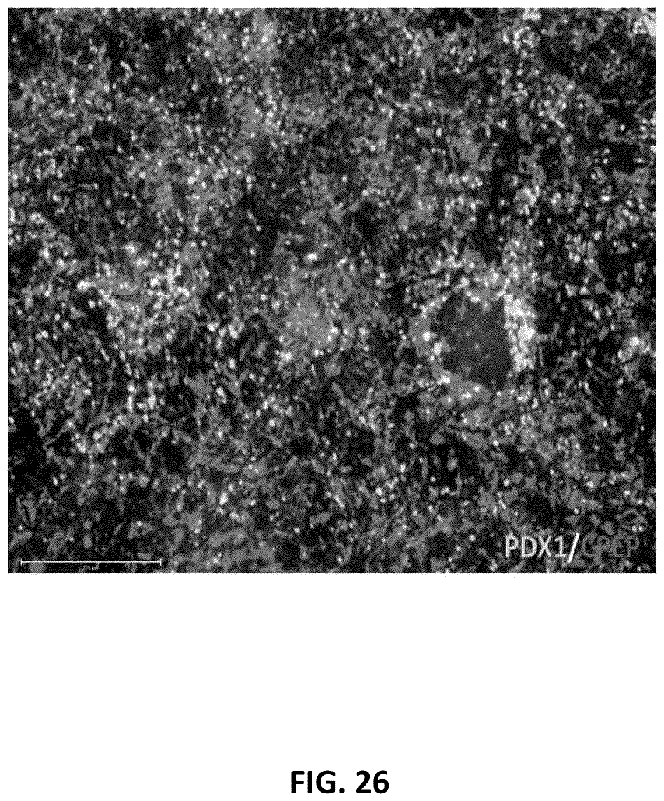

The addition of quercetin to the stage 3 media increased overall endocrine conversion with an overall higher induction of de novo beta cell generation as shown in the immunohistochemical analysis of a day 16 culture shown in FIG. 26. C-peptide staining was performed to distinguish between de novo generation of beta cells and the possible absorption of insulin from the culture media.

### Example 26: Immunohistochemical Validation of the Generation of the De Novo Beta Cells

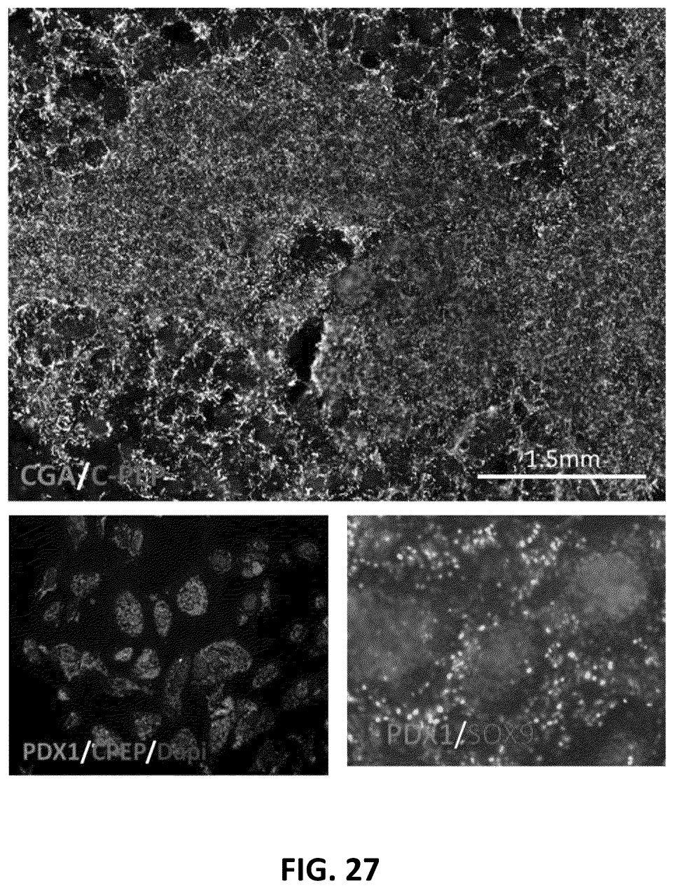

As shown in FIG. 27, cultures were highly homogenous for endocrine cells as reflected in wide field expression of CGA, a general endocrine marker. C-PEP co-staining was consistent with the cells having a beta cell subtype. Areas interspersed with non-endocrine cell types were shown to be a proendocrine trunk progenitor cells as indicated by SOX9 expression.

### Example 27: Functional Analysis of Beta Cells

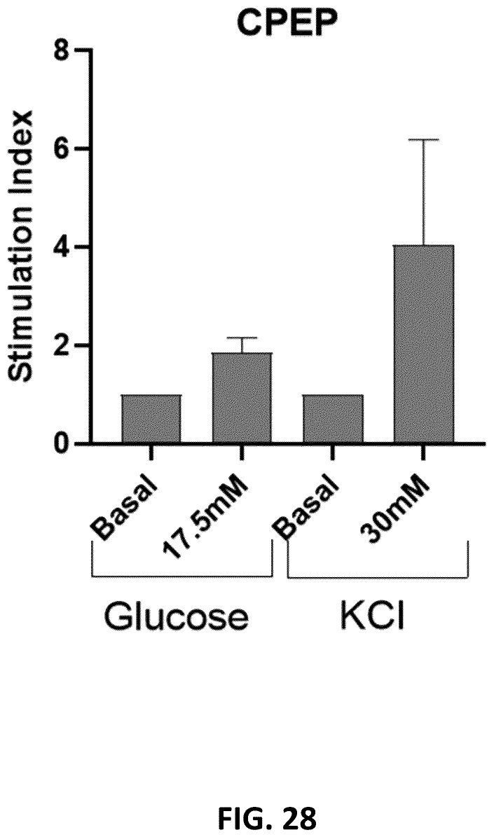

A glucose stimulated insulin secretion assay was performed to evaluate the functional maturity of the de novo beta cells generated. Assay samples had growth media removed and were washed once with PBS to remove any trace of media that may have been previously conditioned with secreted C-PEP. All samples were then incubated in the presence of basal media at 37 degrees Celsius for 15 minutes. Basal media consisted of RPMI containing 3 mM glucose. This media was then changed to either media supplemented with 17.5 mM glucose or 30 mM KCl and incubated for an additional 15 minutes. As shown in FIG. 28, the samples exposed to the 17.5 mM glucose media showed a 2-fold increase in the level of C-Peptide secreted, while the samples exposed to the 30 mM KCl produced C-Peptide levels approximately 4-fold over the basal C-Peptide secretion levels. These results demonstrate that the de novo beta cells response to a glucose challenge fell short of a maximal secretion capacity for the cells, suggesting a limited functional capacity reflective of a more immature cellular state.

### Example 28: Directed Differentiation Beta Cell Protocol is Amenable to Suspension Culture Systems

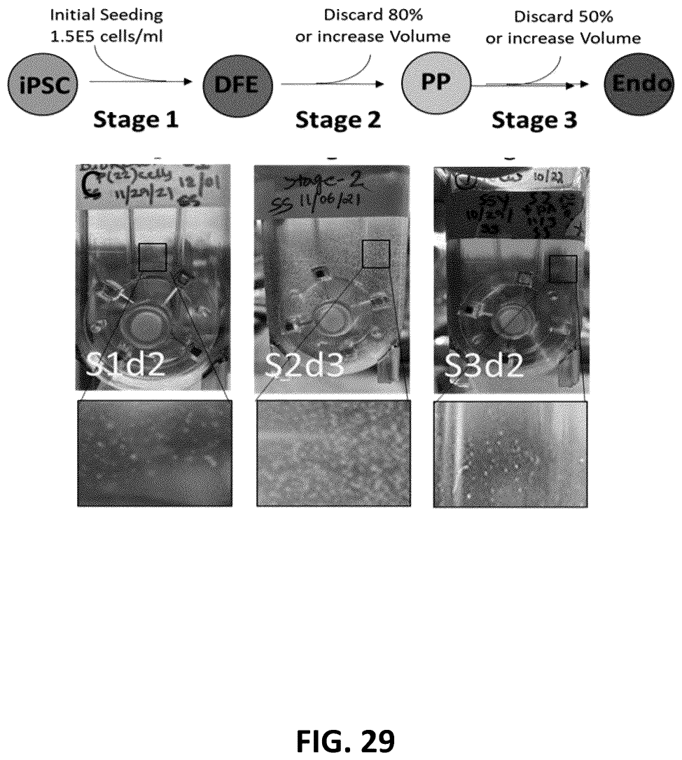

A bioreactor system depicted in FIG. 29 was chosen for application of the iPSC derived DFE protocol to PBS vertical wheel bioreactors. This culture system was ideal because of its scalable nature with bioreactors available ranging in size from 0.1 to 80 liters. Initial efforts at adapting the protocol focused on the stagewise progression of the differentiation process.

### Example 29: Growth and Expansion Occurs Throughout the Differentiation Process

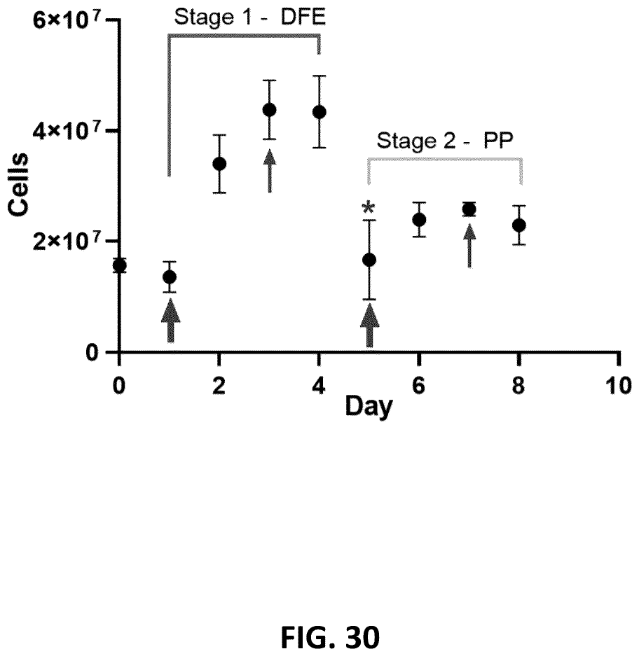

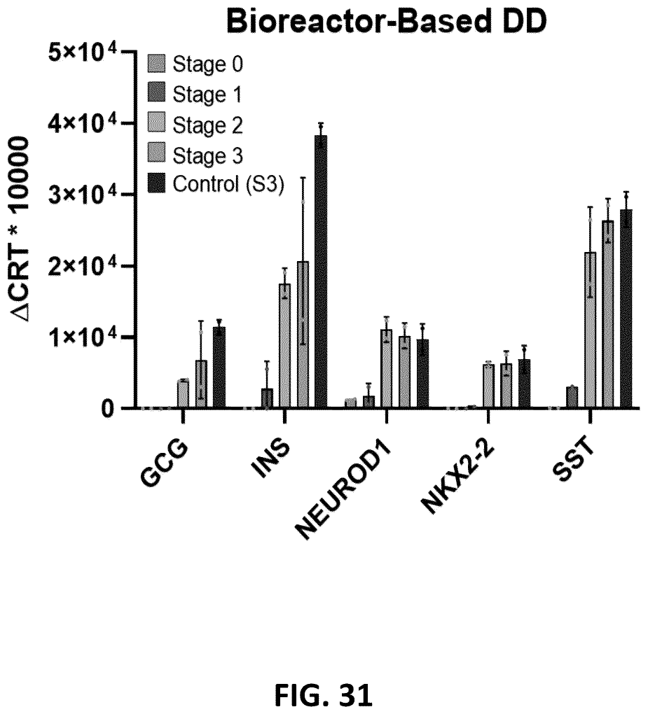

Cultures displayed an initial proliferative phase that tapered off within 4-5 days followed by a declining number of cells surviving the sequential stages of the protocol (FIG. 30). The decreased viability observed and the overall arrest in the differentiation event was attributed to an increase in aggregate size. It is a known problem with bioreactors that as aggregate sizes increase, nutrient availability is limited due to diffusibility limitations. To overcome these limitations, regular disruption of the aggregate state was incorporated in between the discrete stages of the protocol. This maintained a smaller aggregate size, ensuring the availability of nutrients and allowing for the continued proliferative state. A decreased rate of proliferation was observed throughout the differentiation process. Progression from the DFE progenitor to the PP progenitor within the PBS vertical Wheel Bioreactor showed a 5-fold and 3-fold expansion, respectively. This was expected since progenitor cells are highly proliferative while terminally differentiated cells are quiescent.

### Example 30: Equivalent Phenotype Observed Between Bioreactors and Nascent Protocols

The continued growth in the bioreactors resulted in a significant increase in the biomass being produced, with an estimated 20-50 fold expansion capability for the iPSC culture. For the initial establishment of the bioreactor-based protocol, a reduction of biomass throughout the process was used to maintain an optimal cellular density within the bioreactors. To confirm a similar phenotype between cells differentiated in the bioreactors as compared to cells differentiated using the nascent protocol, transcript levels of some key endocrine genes were compared. The endocrine products INS, GCG and SST representative of the pancreatic alpha, beta and delta cells, respectively, were measured throughout the different stages of the bioreactor runs. As expected, these hormones were undetectable in the early stages, but increased significantly by stage 3. Two highly specific beta specific genes NEUROD and NKX2.2 were also measured showing a similar pattern of expression. No significant differences in transcript levels were noted when comparing the bioreactor run to the control culture differentiated on adherent TC conditions.

### Example 31: De Novo Insulin Production within Bioreactors

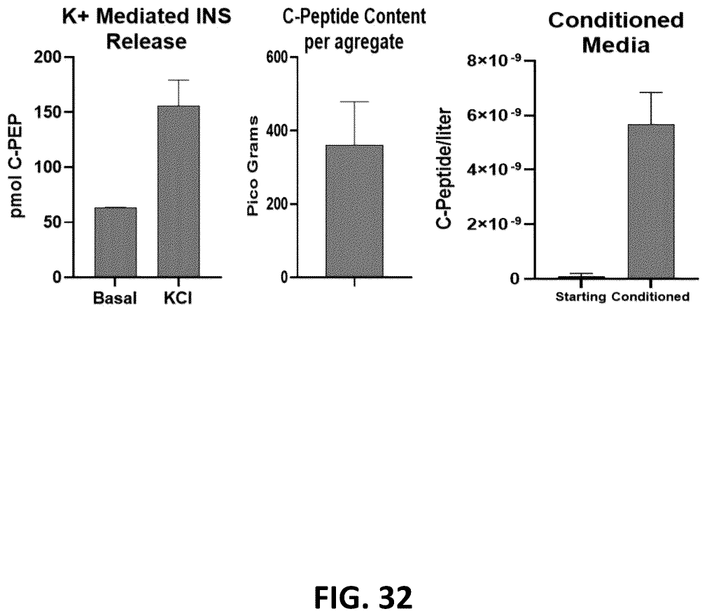

Stage 3 bioreactor runs consistently produced average densities of 100 aggregates/ml with aggregates consisting of approximately 500 cells each. C-peptide was detected in both the stage 3 media used to differentiate the cellular aggregates and within cellular aggregates (FIG. 32). Basal secretion of the insulin content is constant with an immature beta-cell phenotype which has been observed by many other groups. C-peptide was detected at levels of 6.5 ng/l in the media used in the bioreactor. C-peptide is produced when proinsulin is processed to a functioning insulin molecule and is only present when de novo insulin is produced. The C-peptide detection method used is highly specific to human C-peptide and the differentiation media used only contains recombinant insulin lacking this region of the peptide. To further confirm that the C-peptide within the conditioned media is coming from the aggregates control media samples that were not exposed to the differentiating aggregates were analyzed in parallel and showed no detectable levels (FIG. 32). In addition, cell lysis of stage 3 aggregates was assayed for the presence of C-peptide and a cellular content of C-peptide ranging from 250-500 pg per aggregate was detected indicating that iPSCs differentiated within the Bioreactors were both producing and processing insulin.

### Example 32: Additional Characterization of Insulin Producing Cells

In this example, additional experiments were performed to characterize the insulin producing cells.

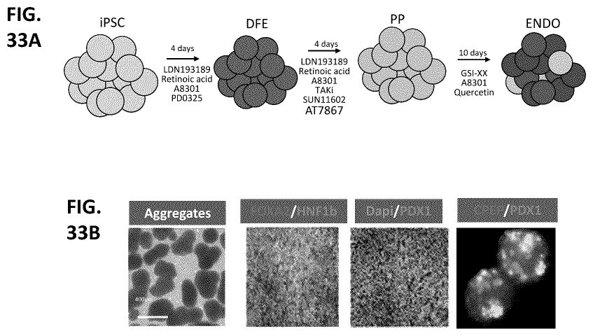

As demonstrated in FIG. 33A-B, bioreactor runs consistently achieved insulin producing cells. iPSC cultures are grown as aggregates to an average size of 150 um diameter. After stage 1 media induction of the dorsal foregut endoderm, a sample of the aggregates was treated with accutase and seeded onto vitronectin. This was followed by staining for the markers FOXA2 and HNF1B to confirm DFE induction within a bioreactor. DFE cultures were then exposed to stage 2 media followed by treated a sample with accutase then seeding onto vitronectin for IHC imaging. Cultures were stained for the pancreatic progenitor marker PDX1. These PP cultures were then exposed stage 3 media and seeded onto Matrigel to preserve them as aggregates. They were stained the following day for the beta-cell specific markers CPEP and PDX1.

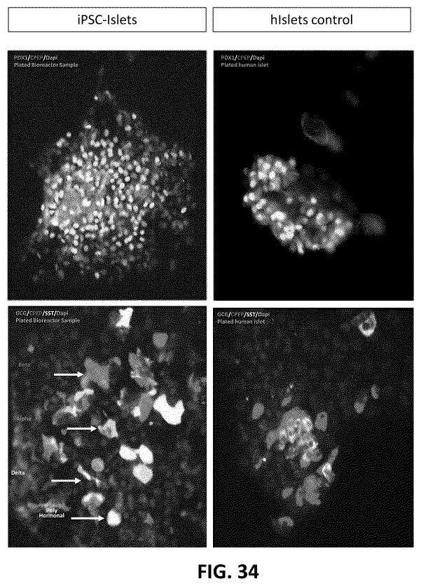

As shown in FIG. 34, bioreactor produced endocrine cells have similar gene expression patterns as compared to human islets. Aggregates produced within a bioreactor run and primary human islets were seeded onto Matrigel for an IHC analysis comparison. Cells were evaluated for PDX1 and CPEP expression patterns, demonstrating similar expression patterns. It should be noted that the iPSC-derivatives PDX1 expression was not limited to the CPEP+ cells, indicating the continued occurrence of pancreatic progenitors within the culture. Looking at the endocrine sub-population, it was noted that all major endocrine sub-types were present within the iPSC-derivatives. In addition to cells expressing CPEP (beta cell), GCG (alpha cell), and SST (delta cell), cells expressing multiple endocrine products within the same cell were noted. These are indicative of immature, not fully committed endocrine cells.

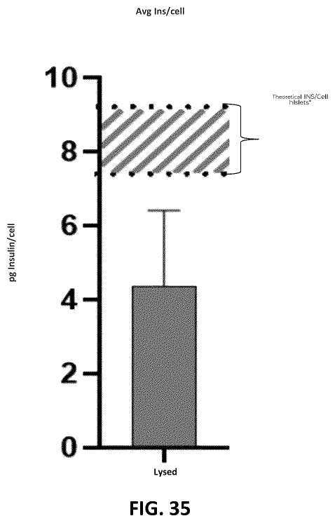

The average insulin content per cell was determined, the results of which are shown in FIG. 35. Aggregates were lysed using TPER and CPEP content was evaluated through ELISA. This was then divided by the estimated number of cells within the aggregate sample and compared to a theoretical estimation of the insulin content within human islets. It was determined that the aggregates produced throughout the bioreactor run contained approximately 50% the level of insulin expected within human islets. Note the conversion between a measurement of CPEP and INS content assumes a 1:1 ratio between CPEP and INS, since they are initially synthesized as a single polypeptide before being processed and stored within vesicles together.

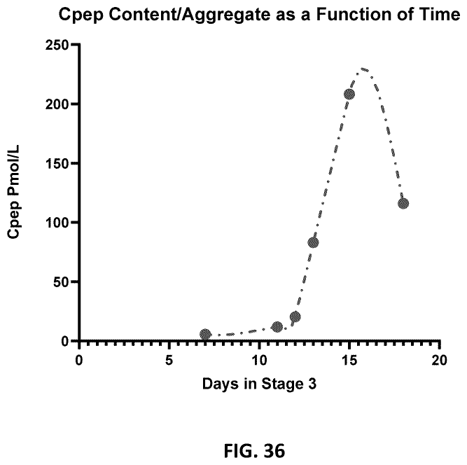

As shown in FIG. 36, insulin production continued for up to two weeks after initial induction. Bioreactor cultures were monitored and sampled along the course of a stage 3 induction. It was observed that the content of CPEP increased up to 14 days at which time the level of CPEP leveled off. This indicated that increasing the length of time that the aggregates were maintained in a stage 3 media increased the overall efficiency of the differentiation event.

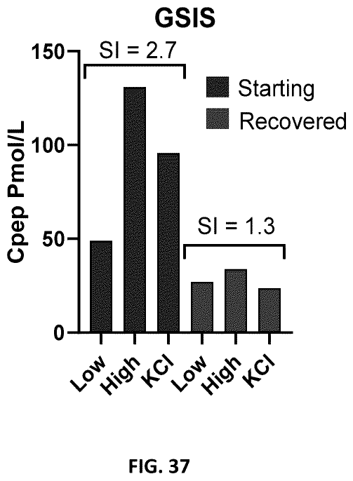

As shown in FIG. 37, glucose stimulated insulin secretion was not maintained after cryopreservation. The iPSC derivatives demonstrate a variable degree of functionality when challenged with glucose influxes. The results showed that the functionality and the overall insulin content of the iPSC derivatives decreased when undergoing cryopreservation, with a decrease from a stimulation index (SI) of 2.7 before freezing to a SI of 1.3 after recovery.

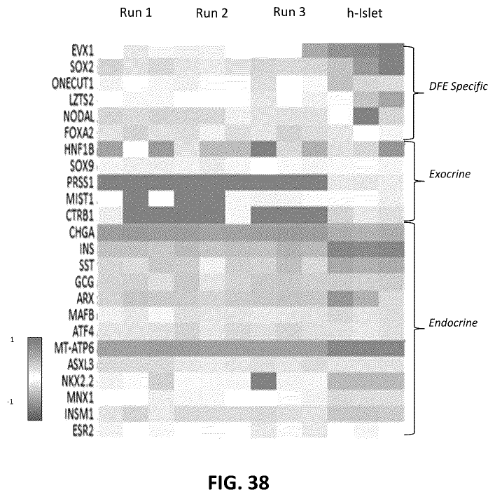

As shown in FIG. 38, bioreactor-based production runs consistently produced insulin-producing cells at levels comparable to human islets. Using three separate bioreactor runs of stage 3 cells, RNA sequencing was performed to evaluate expression patterns representative of different aspects of the differentiation process. It was noted that genes representative of the dorsal foregut endoderm were expressed higher within the iPSC-derivative than they were in the primary islets. This is indicative of the retention of a dorsal phenotype. Notably SOX2, NODAL and FOXA2 expression was still elevated within the iPSC derivatives. Genes representative of the exocrine components of the pancreas were shown to be expressed at higher levels within the primary human islets. This is consistent with small levels of exocrine tissue carrying-over into the extraction process during islet purification. And it is also indicative of the lack of exocrine tissue within the bioreactor-based production of the iPSC-derivatives. Several endocrine-specific genes were evaluated. Notably the iPSC-derivatives consistently showed elevated levels of CHGA (a pan-endocrine marker) indicating a higher level of general endocrine cells, though the actual levels of actual endocrine products were lower as observed from the decreased levels of INS, GCG, and SST expression. Elevated expression of the early alpha cells marker ARX with decreased expression of the mature alpha cell marker MAFB was observed within the iPSC-derivatives. Indicating an early immature alpha cell phenotype within the iPSC-derivatives. A similar lack of markers of a mature phenotype was observed within the iPSC-derived insulin producing cells with decreased expression of NKX2.2, MNX1, INSM1 and ESR2 being observed when compared to human islets.

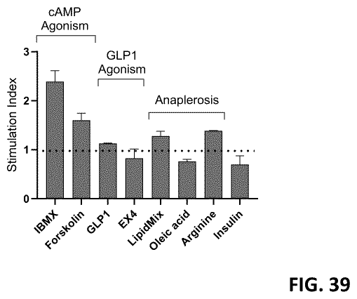

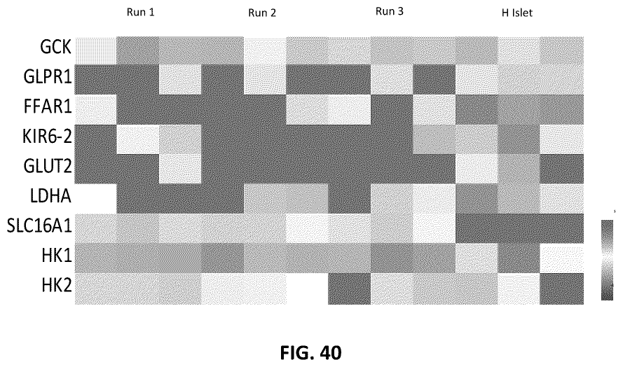

As shown in FIG. 39, the overall functionality of the bioreactor produced insulin secreting cells occurs through cAMP agonism. Assaying the mechanism of insulin secretion for the iPSC derivatives showed that when cAMP agonists were used in conjunction with a GSIS assay, they were able to increase insulin secretion. This increase in insulin release due to cAMP agonism did not occur when the cAMP agonist used were GLP1 or exendin 4 (EX4). Fully mature beta cells respond to incretins such as GLP1 through a cAMP mediated response. The iPSC derivatives are non-responsive to both GLP1 or a commonly used agonist of the GLP1 pathway EX4. It was shown that the receptor for these incretins (GLPR1) had very low expression in the iPSC-derivatives (FIG. 40). The rate of insulin secretion was also directly linked to the rate of mitochondrial respiration, as fully mature beta cells lack LDHA expression, making them only capable of metabolizing glucose through mitochondria respiration. While the LDHA expression of the iPSC derivatives was similar to that of primary islets, anabolic factors that feed directly into the mitochondria, which normally would increase overall mitochondrial respiration, had little effect in increasing insulin secretion (FIG. 39), suggesting that the iPSC derivatives lack the mitochondrial mass found in fully functional beta cells.

RNA sequencing results for gene expression are shown in FIG. 40. Fully functional beta cells have a specific GLUT2 glucose transporter that functions constitutively, which allows for the intracellular glucose concentration to be regulated by the concentration of glucose in the serum. Fully functional beta cells also have a highly specific hexokinase enzyme responsible for phosphorylation of the intracellular glucose forcing it into a glycolytic pathway, this hexokinase, glucokinase (GCK) has a Km with a low binding affinity enabling it to regulate the intracellular concentration of glucose to that of the ideal serum level. While GCK expression is similar between our iPSC-derivatives and primary beta cells, the GLUT2 transporter has lower expression. This limits the ability of the iPSC derivatives to match their intracellular glucose concentration to that of the basal media and consequently blunts their ability to properly respond to glucose fluctuations.

In addition, the iPSC-derivatives have the continued expression of HK1, HK2 and SLC16A1. The down-regulation of these three genes has been shown to be crucial for achieving a fully functional phenotype. The iPSC derivatives also show decreased expression of other crucial maturation markers FFAR1 and KIR6-2. All together this demonstrates that the iPSC-derivatives are not fully functional and have a limited response to glucose.

### Equivalents

Those skilled in the art will recognize or be able to ascertain using no more than routine experimentation, many equivalents of the specific embodiments of the invention described herein. Such equivalents are intended to be encompassed by the following claims

1. A method of generating human FOXA2+HNF1b+ dorsal foregut endodermal cells (DFECs) comprising: culturing human pluripotent stem cells (PSCs) in a culture media comprising a BMP pathway antagonist, a RA pathway agonist, a TGF-β pathway antagonist, and an MEK pathway antagonist on to obtain human FOXA2+HNF1b+ human DFECs.

2. The method of claim 1, wherein the DFECs are further cultured in media comprising a BMP pathway antagonist, a RA pathway agonist, a TGF-β pathway antagonist, a TAK1 pathway antagonist, a bFGF mimetic, and an Akt pathway antagonist to obtain human PTF1A+PDX1+ pancreatic progenitor cells (PPCs).

3. The method of claim 2, wherein the PPCs are further cultured in a culture media comprising a γ-secretase inhibitor, a TGF-β pathway antagonist, and a flavonoid to obtain human INS+PDX+ pancreatic beta cells (PBCs).

4. The method of claim 1, wherein the human pluripotent stem cells are induced pluripotent stem cells (iPSCs).

5. The method of claim 1, wherein the human pluripotent stem cells are embryonic stem cells.

6. The method of claim 1, wherein the human pluripotent stem cells are attached to vitronectin-coated plates during culturing.

7. The method of claim 1, wherein the BMP pathway antagonist is selected from the group consisting of LDN193189, DMH1, DMH2, Dorsomorphin, K02288, LDN214117, LDN212854, folistatin, ML347, Noggin, and combinations thereof.

8. The method of claim 7, wherein BMP pathway antagonist is LDN193189, which is present in the culture media at a concentration of 250 nM.

9. The method of claim 1, wherein the RA pathway agonist is selected from the group consisting of retinoic acid (RA), TTNPB, AM 580, CD 1530, CD 2314, Ch 55, BMS 753, Tazarotene, Isotretinoin, AC 261066, Sr11237, adapalene, EC23, 9-cis retinoic acid, 13-cis retinoic acid, 4-oxo retinoic acid, All-trans Retinoic Acid (ATRA), and combinations thereof.

10. The method of claim 9, wherein the RA pathway agonist is RA, which is present in the culture media at a concentration of 2 nM.

11. The method of claim 1, wherein the TGF-β pathway antagonist is selected from the group consisting of A8301, SB-431542, GW788388, SB525334, TP0427736, RepSox, SD-208, and combinations thereof.

12. The method of claim 11, wherein the TGF-β pathway antagonist is A8301, which is present in the culture media at a concentration of 500 nM.

13. The method of claim 1, wherein the MEK pathway antagonist is selected from the group consisting of PD0325901, Binimetinib (MEK162), Cobimetinib (XL518), Selumetinib, Trametinib (GSK1120212), CI-1040 (PD-184352), Refametinib, ARRY-142886 (AZD-6244), PD98059, U0126, BI-847325, RO 5126766, and combinations thereof.

14. The method of claim 13, wherein the MEK pathway antagonist is PD0325901, which is present in the culture media at a concentration of 250 nM.

15. The method of claim 2, wherein the TAK1 pathway antagonist is selected from the group consisting of Taki ((5Z)-7-Oxozeaenol), Takinib, Dehydoabietic acid, NG25, Sarsasapogenin, and combinations thereof.

16. The method of claim 15, wherein the TAK1 pathway antagonist is Taki ((5Z)-7-Oxozeaenol), which is present in the culture media at a concentration of 500 nM.

17. The method of claim 2, wherein the bFGF mimetic is SUN11602, which is present in the culture media at a concentration of 300 nM.

18. The method of claim 2, wherein the Akt pathway antagonist is selected from the group consisting of AT7867, Sc79, Demethyl-Coclaurine, LM22B-10, YS-49, YS-49 monohydrate, Demethylasterriquinone B1, Recilisib, N-Oleyol glycine, NSC45586 sodium, Periplocin, CHPG sodium salt, Bilobalide, 6-hydroxyflavone, Musk ketone, SEW2871, 8-Prenylnaringenin, Razuprotafib, and combinations thereof.

19. The method of claim 18, wherein the Akt pathway antagonist is AT7867, which is present in the culture media at a concentration of 250 nM.

20. The method of claim 3, wherein the γ-secretase inhibitor is 7-secretase inhibitor XX, which is present in the culture media at a concentration of 100 nM.

21. The method of claim 3, wherein the flavonoid is quercetin, which is present in the culture media at a concentration of 15 μM.

22. A method of generating human FOXA2+HNF1b+ dorsal foregut endodermal cells (DFECs) comprising culturing human pluripotent stem cells in a culture media comprising a BMP pathway antagonist, a RA pathway agonist, a TGF-β pathway antagonist, and an MEK pathway antagonist to obtain the human DFECs.

23. A method of generating human PTFIA+PDX1+ pancreatic progenitor cells (PPCs) comprising:
(a) culturing human pluripotent stem cells in a culture media comprising a BMP pathway antagonist, a RA pathway agonist, a TGF-β pathway antagonist, and an MEK pathway antagonist to obtain human FOXA2+HNF1b+ DFECs; and
(b) further culturing the DFECs in a culture media comprising a BMP pathway antagonist, a RA pathway agonist, a TGF-β pathway antagonist, a TAK1 pathway antagonist, a bFGF mimetic, and an Akt pathway antagonist to obtain human PTF1A+PDX1+PPCs.

24. The method of claim 23, wherein the BMP pathway antagonist is LDN193189, the RA pathway agonist is retinoic acid, the TGF-β pathway antagonist is A8301, the MEK pathway antagonist is PD0325901, the TAK1 pathway antagonist is Taki ((5Z)-7-Oxozeaenol), the bFGF mimetic is SUN11602, and the Akt pathway antagonist is AT7867.

25. The method of claim 24, wherein LDN193189 is present in the culture media at a concentration within a range of 100-400 nM, retinoic acid is present in the culture media at a concentration within a range of 0.5-2 μM, A8301 is present in the culture media at a concentration within a range of 200-1000 nM, PD0325901 is present in the culture media in step (a) at a concentration within a range of 100-400 nM, Taki ((5Z)-7-Oxozeaenol) is present in the culture media at a concentration within a range of 200-1000 nM, SUN11602 is present in the culture media at a concentration within a range of 150-600 nM, and AT7867 is present in the culture media in step (a) at a concentration within a range of 100-400 nM.

26. The method of claim 24, wherein LDN193189 is present in the culture media at a concentration of 250 nM, retinoic acid is present in the culture media at a concentration of 2 μM, A8301 is present in the culture media at a concentration of 500 nM, PD0325901 is present in the culture media at a concentration of 250 nM, Taki ((5Z)-7-Oxozeaenol) is present in the culture media at a concentration of 500 nM, SUN11602 is present in the culture media at a concentration of 300 nM, and AT7867 is present in the culture media in step (a) at a concentration of 250 nM.

27. A method of generating human pancreatic beta cells (PBCs) comprising:
(a) culturing human pluripotent stem cells in a culture media comprising a BMP pathway antagonist, a RA pathway agonist, a TGF-β pathway antagonist, and an MEK pathway antagonist to obtain human dorsal foregut endodermal cells (DFECs);
(b) further culturing the DFECs in a culture media comprising a BMP pathway antagonist, a RA pathway agonist, a TGF-β pathway antagonist, a TAK1 pathway antagonist, a bFGF mimetic, and an Akt pathway antagonist to obtain human pancreatic progenitor cells (PPCs); and
(c) further culturing the human PPCs in a culture media comprising a Notch pathway antagonist, a TGF-β pathway antagonist, and a flavonoid to obtain human pancreatic beta cells (PBCs).

28. The method of claim 27, wherein the BMP pathway antagonist is LDN193189, the RA pathway agonist is retinoic acid, the TGF-β pathway antagonist is A8301, the MEK pathway antagonist is PD0325901, the TAK1 pathway antagonist is Taki ((5Z)-7-Oxozeaenol), the bFGF mimetic is SUN11602, the Akt pathway antagonist is AT7867, the Notch pathway antagonist is 7-secretase inhibitor XX (GSI-XX), and the flavonoid is quercetin.

29. The method of claim 28, wherein LDN193189 is present in the culture media in steps (a) and (b) at a concentration within a range of 100-400 nM, retinoic acid is present in the culture media in steps (a) and (b) at a concentration within a range of 0.5-2 μM, A8301 is present in the culture media in steps (a)-(c) at a concentration within a range of 200-1000 nM, PD0325901 is present in the culture media in step (a) at a concentration within a range of 100-400 nM, Taki ((5Z)-7-Oxozeaenol) is present in the culture media in step (b) at a concentration within a range of 200-1000 nM, SUN11602 is present in the culture media in step (b) at a concentration within a range of 150-600 nM, AT7867 is present in the culture media in step (b) at a concentration within a range of 100-400 nM, GSI-XX is present in the culture media in step (c) at a concentration within a range of 50-200 nM, and quercetin is present in the culture media in step (c) at a concentration within a range of 5-30 μM.

30. The method of claim 28, wherein LDN193189 is present in the culture media in steps (a) and (b) at a concentration of 250 nM, retinoic acid is present in the culture media in steps (a) and (b) at a concentration of 2 μM, A8301 is present in the culture media in steps (a)-(c) at a concentration of 500 nM, PD0325901 is present in the culture media in step (a) at a concentration of 250 nM, Taki ((5Z)-7-Oxozeaenol) is present in the culture media in step (b) at a concentration of 500 nM, SUN11602 is present in the culture media in step (b) at a concentration of 300 nM, AT7867 is present in the culture media in step (b) at a concentration within a range of 250 nM, GSI-XX is present in the culture media in step (c) at a concentration of 100 nM, and quercetin is present in the culture media in step (c) at a concentration of 15 μM.

31. A culture media for obtaining human dorsal foregut endoderm cells (DFECs) comprising culture media comprising a BMP pathway antagonist, a RA pathway agonist, a TGF-β pathway antagonist, and an MEK pathway antagonist.

32. A culture media for obtaining human pancreatic progenitor cells (PPCs) comprising a BMP pathway antagonist, a RA pathway agonist, a TGF-β pathway antagonist, a TAK1 pathway antagonist, a bFGF mimetic, and an Akt pathway antagonist.

33. A culture media for obtaining human pancreatic beta cells (PBCs) comprising a Notch pathway antagonist, a TGF-β pathway antagonist, and a flavonoid.

34. An isolated cell culture of human FOXA2+HNF1b+ dorsal foregut endoderm cells (DFECs), the culture comprising human DFECs cultured in the culture media of claim 31.

35. An isolated cell culture of human PTF1A+PDX1+ pancreatic progenitor cells (PPCs), the culture comprising human PPCs cultured in the culture media of claim 32.

36. An isolated cell culture of human INS+PDX+ pancreatic beta cells (PBCs), the culture comprising human PBCs cultured in the culture media of claim 33.

37. Human FOXA2+HNF1b+ dorsal foregut endodermal cells (DFECs) generated by the method of claim 1.

38. Human PTF1A+PDX1+ pancreatic progenitor cells (PPCs) generated by the method of claim 2.

39. Human INS+PDX+ pancreatic beta cells (PBCs) generated by the method of claim 3.
